(19) United States
(12) Patent Application Publication
(10) Pub. No.: US 2010/0209419 A1
(43) Pub. Date: Aug. 19, 2010

(54) Method and formulation for treating adverse biological conditions

(76) Inventors: Rajiv Bhushan, Palo Alto, CA (US); Jerry B. Gin, Sunnyvale, CA (US); Amit Goswamy, Los Gatos, CA (US)

Correspondence Address: MINTZ, LEVIN, COHN, FERRIS, GLOVSKY AND POPEO, P.C, One Financial Center, BOSTON, MA, 02111, US

(21) Appl. No.: 12/583,304

(22) Filed: Aug. 17, 2009

Related U.S. Application Data
(60) Provisional application No. 61/189,060, filed on Aug. 15, 2008.

(51) Int. Cl. A61K 39/395; A61K 31/7088; A61K 38/16; A61K 38/43; A61P 43/00; A61K 31/715

(52) U.S. Cl. 424/133.1; 514/44 R; 514/44 A; 514/12; 424/94.1; 424/130.1; 514/54

(57) ABSTRACT

A method for treatment of adverse biological conditions is provided, wherein a biologically active agent such as a macromolecular biomolecule, e.g., a nucleic acid or a peptidic compound, is administered to a subject in need of such treatment in a formulation containing a transport enhancer having the structure of formula (I)

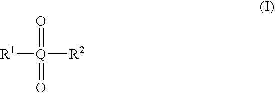

wherein Q, R¹, and R² are as defined herein. Methylsulfonylmethane (MSM) is a representative and preferred transport enhancer. Formulations are also provided.

### CROSS-REFERENCE TO RELATED APPLICATIONS

This application claims priority under 35 U.S.C. §119(e) to Provisional U.S. Patent Application Ser. No. 61/189,060, filed Aug. 15, 2008, the disclosure of which is incorporated by reference herein.

### BACKGROUND

Disease produces complex biological responses within an affected organism. Disease conditions may result from harmful environmental stimuli, such as oxidative stress, irritants, and pathogens, and/or from problems associated with atypical genetic factors. For instance, the aberrant regulation of endogenous genes and/or protein production can lead to such adverse conditions as cancer, diabetes, liver disease, kidney disease, anemia, and the like. In such instances, treatment modalities that are capable of restoring normal protein production mechanisms are useful in ameliorating the harmful consequences of genetic abnormalities.

Many modalities proposed for the treatment of such disease conditions include the delivery of biologically active agents to various cells of the body. For instance, techniques have been developed for the delivery of macromolecular agents, including genetic material (e.g., nucleotide-based drugs) and peptidic molecules (e.g., proteins, protein fragments, peptides, etc.), to the extracellular milieu and/or into the cell for the treatment of a disease state. These techniques may be useful for regulation of gene expression, for regulation of peptide or protein production, for diagnostic purposes, and/or in the analysis of intracellular events that result from the transport of a variety of different substances into a cell.

Such techniques include gene, protein, and anti-sense therapy. Gene therapy involves the insertion of a gene into the nucleus of a cell and/or genome of a subject for the replacement of a mutant or non-functioning allele with a normal, functional allele.

Protein therapy involves the delivery of a protein to a cell surface and/or into the cell where the cell does not effectively produce the protein in an efficient and/or effective manner such that a diseased condition results. Protein therapy manipulates the cell by introducing a protein to the cell so as to shift the endogenous cell dynamics towards that of a healthy and/or productive cell.

Another technique similar to gene therapy is anti-sense therapy. Anti-sense therapy is a technique employed for the treatment of genetic disorders and/or infections when the sequence of a particular gene is known to be causative of a particular disease. Anti-sense therapy involves the synthesis of an anti-sense strand of nucleic acid (e.g., DNA, RNA, or a chemical analogue thereof) and the introduction of that nucleic acid into the nucleus of a target cell. Once in the target cell the anti-sense strand of nucleic acid may bind a splicing site on pre-messenger RNA (mRNA) and modify the exon content thereof, or it may bind mRNA produced by a gene within the cell and thereby inactivate it. Because mRNA has to be single stranded for it to be translated, the binding of the anti-sense nucleic acid to its respective target effectively results in the inactivation of the relevant gene.

One aspect that all of these techniques have in common is the delivery of the biologically active agent to, and in some instances, through the cell. Several techniques have, therefore, been developed in an effort to improve the efficiency with which biological agents such as nucleic acids and proteins can be delivered to cells. Representative examples of such techniques include, without limitation, coprecipitation of the biologically active agent with calcium phosphate or DEAE-dextran, electroporation, biolistics, and vector-mediated transfection and transduction. These techniques are designed to enable the biologically active agent to penetrate the plasma membrane of a cell and thereby enter the cell and/or an organelle thereof. All of these techniques, however, suffer from low transfer efficiency and a high probability of cell death. Other methods employ a conjugate of a virus-related substance with a nucleic acid of interest. Viral conjugates are difficult to work with, however, and the use of virus components is risky. Further, such techniques are limited by the inability of gene transfer vectors to transfer the biologically active agents into the cytoplasm or nuclei of cells in the subject to be treated without affecting the subject's genome or altering the biological properties of the active agent.

Consequently, a need remains for an efficient, low-risk means for delivering a biologically active agent to a target cell for the treatment of a disease or other adverse condition. The present methods and formulations meet these and other needs in the art by providing a means for the delivery of a beneficial agent of interest, e.g., a biologically active agent, through the tissues of the body, to a prospective site of delivery, for transfer of the beneficial agent from the extracellular milieu into the cytoplasm of the cell, and, where advantageous, into cellular nuclei.

### SUMMARY OF THE INVENTION

In one aspect of the invention, a method is provided for treating an adverse physiological condition in a subject. The method involves administering to the subject an effective amount of a formulation composed of a therapeutically effective amount of a biologically active agent and an effective transport enhancing amount of a transport enhancer having the formula (I)

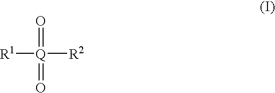

wherein R¹ and R² are independently selected from C₁-C₆ alkyl, C₁-C₆ heteroalkyl, C₆-C₁₄ aralkyl, and C₂-C₁₂ heteroaralkyl, any of which may be substituted, and Q is S or P, wherein the transport enhancer is present in an amount effective to facilitate transport of the biologically active agent such that the biologically active agent is delivered in an amount effective to treat the adverse physiological condition. The transport enhancing agent may be, for example, methylsulfonylmethane (also referred to as methylsulfone, dimethylsulfone, and DMSO₂), and the biologically active agent is generally, although not necessarily, a biomolecule such as a peptidic compound, a nucleotidic compound, a saccharide, a lipidic moiety, or the like.

The invention additionally provides such a method adapted for the treatment of conditions associated with the overexpression of a protein or a protein deficiency, and for the treatment of other specific adverse conditions such as cancer, diabetes, liver disease, kidney disease, anemia, and the like.

The invention further provides formulations for use in the aforementioned methods.

Other features and advantages of the invention will be apparent from the following detailed description and claims.

### BRIEF DESCRIPTION OF THE DRAWINGS

The patent or application contains at least one drawing executed in color. Copies of this patent or patent application publication with color drawings will be provided by the Office upon request and payment of the necessary fee.

According to common practice, the various features of the drawings may not be presented to-scale. Rather, the dimensions of the various features may be arbitrarily expanded or reduced for clarity. Included in the drawings are the following figures:

FIG. 1 is a photograph of a neuroblastoma cell taken with a confocal microscope and illustrates the extent to which a dsRNA-FITC is delivered into the cell in the absence of MSM, with an incubation time of four hours, as described in Example I.

FIG. 2 is a photograph of a neuroblastoma cell taken with a confocal microscope and illustrates the extent to which a dsRNA-FITC is delivered into the cell when combined with MSM, with an incubation time of four hours, also as described in Example I.

FIG. 3A is a photograph of a neuroblastoma cell taken with a confocal microscope and illustrates the extent to which a dsRNA-FITC is delivered into the cell when combined with MSM, when the MSM is premixed with the dsRNA-FITC for 30 minutes, with an incubation time of four hours, also as described in Example I.

FIG. 3B is a photograph of three wells, panels A, B, and C of a neuroblastoma cell, taken with a confocal microscope, illustrating the extent to which a dsRNA-FITC is delivered into the cell when the dsRNA is delivered in the absence of MSM, with an incubation time of about 15 hours, also as described in Example I.

FIG. 4 is a photograph of three wells, panels A, B, and C of a neuroblastoma cell, taken with a confocal microscope, illustrating the extent to which a dsRNA-FITC is delivered into the cell when combined with MSM, when the MSM is premixed with the dsRNA-FITC for 30 minutes, with an incubation time of about 15 hours, also as described in Example I.

FIG. 5 is a photograph of three wells, panels A, B, and C of a neuroblastoma cell, taken with a confocal microscope, illustrating the extent to which a dsRNA-FITC is delivered into the cell in the presence of MSM, when the MSM is premixed with the RNA-FTC for 60 minutes, with an incubation time of about 15 hours, also as described in Example I.

FIG. 6 is a photograph of four wells, panels A, B, C, and D of a neuroblastoma cell, taken with a fluorescent microscope, showing the extent to which a CY3-dsRNA (Red Nuclear stain, Syto16:green stain) is delivered into the cell in the absence of MSM, with an incubation time of about 15 hours, as described in Example II.

FIG. 7 is a photograph of four wells, panels A, B, C, and D of a neuroblastoma cell, taken with a fluorescent microscope, illustrating the extent to which a CY3-dsRNA (Red Nuclear stain, Syto16:green stain) is delivered into the cell in the presence of MSM, with an incubation time of about 15 hours, also as described in Example II.

FIG. 8 is a photograph of four wells, panels A, B, C, and D of a neuroblastoma cell, taken with a fluorescent microscope, illustrating the extent to which a CY3-dsRNA (Red Nuclear stain, Syto16:green stain) is delivered into the cell in the presence of MSM, with an incubation time of about 15 hours, also as described in Example II. The photographs are identical to those in FIG. 7, but magnified.

FIG. 9 is a magnified, enlarged version of FIG. 8, indicating the location of CY3-dsRNA in single cells.

FIG. 10 is a photograph of four panels (A, B, C, and D) of six slides of SH-SY5Y cells taken with a fluorescent microscope, showing the delivery of a secondary antibody conjugated to Texas red into the SH-SY5Y cells (counterstained with DAPI) where the dsRNA is delivered in the absence or presence of MSM, as described in Example III. Magnification was at 200×. Incubation time was one hour.

FIG. 11 is a photograph of a single panel of SH-SY5Y cells taken with a fluorescent microscope, showing the delivery of a secondary antibody conjugated to Texas red into the SH-SY5Y cells (counterstained with DAPI) where the dsRNA is delivered in the presence of MSM, as described in Example III. Incubation time was one hour. Before adding the AB and/or MSM into the cell culture medium the AB and MSM were mixed well and incubated at 37 degrees C. for 30 mins. Magnification was at 400×.

FIG. 12 is a photograph of four wells, panels A, B, C, and D of a neuroblastoma cell, taken with a fluorescent microscope, showing the delivery of a CY3-dsRNA (Red Nuclear stain, Syto16:green stain) into the cell where the dsRNA is delivered in the absence or presence of MSM, as described in Example IV. Incubation time was about 15 hours. Magnification was at 400×.

FIG. 13 is a photograph of four wells, panels A, B, C, and D of a neuroblastoma cell, taken with a fluorescent microscope, showing the delivery of a CY3-dsRNA (Red Nuclear stain, Stylo16:green stain) into the cell where the dsRNA is delivered in the absence or presence of MSM, as described in Example TV. Incubation time was about 15 hours. Magnification was at 400×.

FIG. 14 shows the relative quantities of ³H cyclosporin in the aqueous and vitreous of rats' eyes obtained with and without MSM, as described in Example V.

FIG. 15 also illustrates the relative quantities of ³H cyclosporin delivered into the aqueous and the vitreous with and without MSM, and further illustrates the relative quantities delivered into the retina with MSM (“CM-Re”) and without MSM (“CP-Re”).

FIG. 16 is a graph illustrating the amount of cyclosporin delivered to the aqueous in the presence and absence of MSM, with the 10 minute, 15 minute, and 30 minute data points shown, also as described in Example V.

FIG. 17, similarly, is a graph illustrating the amount of cyclosporin delivered to the vitreous in the presence and absence of MSM, with the 10 minute, 15 minute, and 30 minute data points shown, also as described in Example V.

FIG. 18, similarly, is a graph illustrating the amount of cyclosporin reaching the retina in the presence and absence of MSM, with the 10 minute, 15 minute, and 30 minute data points shown. When the cyclosporin is administered in an MSM-containing formulation, it should be noted that the amount of cyclosporin reaching the retina increases with time.

FIG. 19 illustrates the relative levels of bevacizumab in the retina/choroid of rats obtained with and without MSM, using a single dose and evaluating bevacizumab levels after 16 hours, as described in Example VI.

FIG. 20 illustrates bevacizumab levels in the vitreous humor following multiple doses of the control (no MSM) and experimental (MSM-containing) solutions, with bevacizumab levels evaluated after 4 hours, as described in Example VI.

### DETAILED DESCRIPTION OF THE INVENTION

### Terminology and Overview

It is to be understood that unless otherwise indicated this invention is not limited to particular embodiments described, as such may vary. It is also to be understood that the terminology used herein is for the purpose of describing particular embodiments only, and is not intended to be limiting. Unless defined otherwise, all technical and scientific terms used herein have the same meaning as commonly understood by one skilled in the art to which this invention belongs.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, various publications, patents and published patent applications are cited. The inventions of these publications, patents and published patent applications referenced in this application are hereby incorporated by reference in their entireties into the present invention. Citation herein of a publication, patent, or published patent application is not an admission the publication, patent, or published patent application is prior art.

As used herein and in the appended claims, the singular forms “a,” “and,” and “the” include plural referents unless the context clearly dictates otherwise. Thus, for example, “a transport enhancer” encompasses a plurality of transport enhancers as well as a single transport enhancer, reference to “a beneficial agent” includes reference to two or more beneficial agents as well as a single beneficial agent, and so forth.

In this specification and in the claims that follow, reference will be made to a number of terms, which shall be defined to have the following meanings:

“Optional” or “optionally present”—as in an “optional substituent” or an “optionally present additive” means that the subsequently described component (e.g., substituent or additive) may or may not be present, so that the description includes instances where the component is present and instances where it is not.

By “pharmaceutically acceptable” is meant a material that is not biologically or otherwise undesirable, e.g., the material may be incorporated into a formulation of the invention without causing any undesirable biological effects or interacting in a deleterious manner with any of the other components of the dosage form formulation. However, when the term “pharmaceutically acceptable” is used to refer to a pharmaceutical excipient, it is implied that the excipient has met the required standards of toxicological and manufacturing testing and/or that it is included on the Inactive Ingredient Guide prepared by the U.S. Food and Drug Administration. As explained in further detail infra, “pharmacologically active” (or simply “active”) as in a “pharmacologically active” derivative or analog refers to derivative or analog having the same type of pharmacological activity as the parent agent.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of an undesirable condition or damage. Thus, for example, “treating” a subject involves prevention of an adverse condition in a susceptible individual as well as treatment of a clinically symptomatic individual by inhibiting or causing regression of the condition.

The term “biologically active agent” (or “active agent”) refers to any chemical compound, complex or composition that exhibits a desirable effect in the biological context, i.e., when administered to a subject or introduced into cells or tissues in vitro. The term includes pharmaceutically acceptable derivatives of those active agents specifically mentioned herein, including, but not limited to, salts, esters, amides, prodrugs, active metabolites, isomers, analogs, crystalline forms, hydrates, and the like. When the term “biologically active agent” is used, or when a particular biologically active agent is specifically identified, it is to be understood that pharmaceutically acceptable salts, esters, amides, prodrugs, active metabolites, isomers, analogs, etc. of the agent are intended as well as the agent per se.

By an “effective” amount or a “therapeutically effective” amount of an active agent is meant a nontoxic but sufficient amount of the agent to provide a beneficial effect. The amount of active agent that is “effective” will vary from subject to subject, depending on the age and general condition of the individual, the particular active agent or agents, and the like. Unless otherwise indicated, the term “therapeutically effective” amount as used herein is intended to encompass an amount effective for the prevention of an adverse condition and/or the amelioration of an adverse condition, i.e., in addition to an amount effective for the treatment of an adverse condition.

As will be apparent to those of skill in the art upon reading this invention, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

Unless otherwise indicated, the invention is not limited to specific formulation components, modes of administration, biologically active agents, manufacturing processes, or the like, as such may vary.

The present invention provides methods and formulations for the treatment of adverse physiological conditions by enhancing the transport of a biologically active agent, particularly a biomolecule, to ensure that an effective amount of the agent reaches the intended target within the body. Depending on the biomolecule and the nature of the adverse condition, it may be that a formulation of the invention functions to ensure delivery of a biologically active agent to the surface of a cell. In other instances, it may be that the biologically active agent must penetrate the cell membrane for a benefit to come about, and the formulations and methods of the invention facilitate such penetration. In certain embodiments, the transport enhancer functions to facilitate the penetration of the active agent into various organelles of the cell, such as the cell's nucleus. Accordingly, the invention further includes methods and formulations for facilitating the translocation of a biologically active agent across a biological membrane of a cell, in, for instance, the treatment of an adverse biological condition.

The Transport Enhancer:

The transport enhancer is selected to facilitate the transport of a biologically active agent, particularly a biomolecule, and most typically a macromolecular biomolecule, through the vessels, tissues, extra-cellular matrices, cell membranes, and/or organelles of a body. An “effective amount” of the transport enhancer represents an amount and concentration within a formulation of the invention that is sufficient to provide a measurable increase in the penetration of a biologically active agent through one or more of the vessels, tissues, extracellular matrices, plasma membranes, cells and/or organelles of the body than would otherwise be the case without the inclusion of the transport enhancer within the formulation.

In certain instances, the transport enhancer may be present in a formulation of the invention in an amount that ranges from about 0.01 wt. % or less to about 10 wt. % or more, typically in the range of about 0.1 wt. % to about 10 wt. %, more typically in the range of about 1 wt. % to about 6 wt. %, and most typically in the range of about 2 wt. % to about 4 wt. %, for instance, 3 wt. %.

The transport enhancer is generally of the structural formula (I)

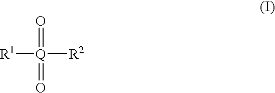

wherein R¹ and R² are independently selected from C₁-C₆ alkyl (preferably C₁-C₃ alkyl), C₁-C₆ heteroalkyl (preferably C₁-C₃ heteroalkyl), C₆-C₁₄ aralkyl (preferably C₆-C₈ aralkyl), and C₂-C₁₂ heteroaralkyl (preferably C₄-C₁₀ heteroaralkyl), any of which may or may not be substituted, and Q is S or P. Compounds wherein Q is S and R¹ and R² are C₁-C₃ alkyl are particularly preferred, with methylsulfonylmethane representing the optimal transport enhancer.

The phrase “having the formula” or “having the structure” is not intended to be limiting and is used in the same way that the term “comprising” is commonly used. With respect to the above structure, the term “alkyl” refers to a linear, branched, or cyclic saturated hydrocarbon group containing 1 to 6 carbon atoms, such as methyl, ethyl, n-propyl, isopropyl, n-butyl, isobutyl, t-butyl, cyclopentyl, cyclohexyl and the like. If not otherwise indicated, the term “alkyl” includes unsubstituted and substituted alkyl, wherein the substituents may be, for example, halo, hydroxyl, sulfhydryl, alkoxy, acyl, etc. The term “alkoxy” intends an alkyl group bound through a single, terminal ether linkage; that is, an “alkoxy” group may be represented as —O-alkyl where alkyl is as defined above. The term “aryl” refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 14 carbon atoms. Exemplary aryl groups are contain one aromatic ring or two fused or linked aromatic rings, e.g., phenyl, naphthyl, biphenyl, diphenylether, diphenylamine, benzophenone, and the like. “Aryl” includes unsubstituted and substituted aryl, wherein the substituents may be as set forth above with respect to optionally substituted “alkyl” groups. The term “aralkyl” refers to an alkyl group with an aryl substituent, wherein “aryl” and “alkyl” are as defined above. Preferred aralkyl groups contain 6 to 14 carbon atoms, and particularly preferred aralkyl groups contain 6 to 8 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The term “acyl” refers to substituents having the formula —(CO)-alkyl, —(CO)-aryl, or —(CO)-aralkyl, wherein “alkyl,” “aryl, and “aralkyl” are as defined above. The terms “heteroalkyl” and “heteroaralkyl” are used to refer to heteroatom-containing alkyl and aralkyl groups, respectively, i.e., alkyl and aralkyl groups in which one or more carbon atoms is replaced with an atom other than carbon, e.g., nitrogen, oxygen, sulfur, phosphorus or silicon, typically nitrogen, oxygen or sulfur.

Suitable transport enhancers include methylsulfonylmethane (MSM; also referred to as methyl sulfone) and/or combinations of MSM with dimethylsulfoxide (DMSO). MSM is an odorless, highly water-soluble (34% w/v at 79° F.) white crystalline compound with a melting point of 108-110° C. and a molecular weight of 94.1 g/mol. MSM is thought to serve as a multifunctional agent herein, insofar as the agent not only increases the permeability of biological membranes such as cell membranes, but may also facilitate the transport of one or more formulation components throughout the layers of the skin (i.e., epidermis, dermis and subcutaneous fat layers), as well as across mucus membranes, endothelial layers, and the like. Furthermore, MSM per se is known to provide medicative effects, and can serve as an anti-inflammatory agent as well as an analgesic. MSM also acts to improve oxidative metabolism in biological tissues, and is a source of organic sulfur, which may assist in the reduction of scarring. MSM additionally possesses beneficial solubilization properties, in that it is soluble in water, as noted above, but exhibits both hydrophilic and hydrophobic properties because of the presence of polar S═O groups and nonpolar methyl groups. The molecular structure of MSM also allows for hydrogen bonding with other molecules, i.e., between the oxygen atom of each S═O group and hydrogen atoms of other molecules, and for formation of van der Waals associations, i.e., between the methyl groups and nonpolar (e.g., hydrocarbyl) segments of other molecules.

The methods and formulations herein may involve use of two or more transport enhancers used in combination. For example, a formulation can contain dimethylsulfoxide (DMSO) in addition to MSM. Since MSM is a metabolite of DMSO (i.e., DMSO is enzymatically converted to MSM), incorporating DMSO into an MSM-containing formulation of the invention will tend to gradually increase the fraction of MSM in the formulation. If DMSO is added to a formulation of the invention as a secondary transport enhancer, the amount is preferably in the range of about 1.0 wt. % to 2.0 wt. % of the formulation, and the weight ratio of MSM to DMSO is typically in the range of about 1:1 to about 50:1.

Biologically Active Agents and Adverse Conditions to be Treated:

The biologically active agents used in conjunction with the present methods and formulations are preferably, although not necessarily, biomolecules. The term “biomolecule” as used herein refers to any organic molecule, whether naturally occurring, recombinantly produced, or chemically synthesized in whole or in part, that is, was, or can be a part of a living organism. The term encompasses, for example, nucleotides, amino acids, fatty acids, and monosaccharides, as well as oligomeric and polymeric species, including, without limitation: nucleic acids, including oligonucleotides and polynucleotides; peptidic molecules such as oligopeptides, polypeptides, and proteins; saccharides such as disaccharides, oligosaccharides, polysaccharides, mucopolysaccharides, and peptidoglycans (peptido-polysaccharides); lipidic oligomers and polymers such as lipopolysaccharides, and the like. The term also encompasses ribosomes, enzyme cofactors, and the like.

Preferred biomolecules herein are macromolecular, meaning that the biomolecule, or “macromolecule,” in this case, has a molecular weight of at least 300, preferably at least 700, more preferably at least 1000, or at least 2000, or is a dimer, oligomer, or polymer. Some macromolecules that can be delivered using the present methods and formulations, have molecular weights on the order of 100,000 or more, e.g., bevacizumab (Avastin®, Genentech/Roche), which has a molecular weight of approximately 149 kD.

In one embodiment, the active agent is a nucleic acid. A nucleic acid, as that term is used herein, refers generally to a macromolecule composed of at least two monomeric nucleotides, where the nucleotide is an N-glycoside of a purine or pyrimidine base, typically adenine, thymine, cytosine, guanine, or uracil, optionally protected, for example, with a protecting group such as an acetyl, difluoroacetyl, trifluoroacetyl, isobutyryl, or benzoyl moiety. Nucleic acids may also contain one or more nucleotide analogs, modified nucleotides, and/or nucleotides substituted with a sugar molecule other than the conventional D-ribose or 2-deoxy-D-ribose component (hereinafter referred to as ribose and 2-deoxyribose, respectively). Nucleotide analogs and modified nucleotides may, for example, include modifications on the sugar moiety, e.g., wherein one or more of the hydroxyl groups are replaced with halogen atoms or aliphatic groups, or are functionalized as ethers, amines, or the like. Other suitable nucleotidic structures are known to those skilled in the art and are described in the pertinent texts and literature.

Nucleic acids herein thus include polydeoxynucleotides (2-deoxyribose), polyribonucleotides (containing ribose), polynucleotides containing modified nucleotides, protected nucleotides, and/or nucleotide analogs, other polymers containing normucleotidic backbones (e.g., containing methylphosphonate, phosphotriester, phosphoramidate, phosphorothioate, etc. linkages), providing that the polymer contain nucleotidic bases in a configuration that allows for base pairing and base stacking, such as is found in DNA and RNA. Nucleic acids herein include double- and single-stranded DNA (dsDNA and sDNA, respectively), as well as double- and single-stranded RNA (dsRNA and sRNA, respectively).

With ribonucleic acids, i.e., when the biologically active agent is an RNA molecule, the RNA may be a messenger RNA (mRNA), a transfer RNA (tRNA), a ribosomal RNA (rRNA), an interfering RNA (iRNA), a minor interfering RNA (miRNA), a small interfering RNA (siRNA), a small nuclear RNA (snRNA), and/or an anti-sense RNA (aRNA).

With deoxyribonucleic acids, i.e., DNA molecules, the DNA may be complementary DNA (cDNA), plasmid DNA, linear DNA, chromosomal DNA, and viral DNA. In certain instances, the nucleic acid comprises a sequence containing a single nucleotide polymorphism (“SNP”) or an expressed sequence tag (“EST”). The nucleic acid may also be an aptamer, either a DNA aptamer or an RNA aptamer. Nucleic acids having more than two strands are also contemplated, including three-stranded DNA and four-stranded DNA.

A formulation herein containing a nucleic acid as the active agent may be administered to a subject so as to treat an adverse condition caused by a genetic defect. For instance, a formulation of the invention containing a transport enhancer, such as MSM, and a nucleic acid may be administered to treat an adverse condition caused by the over-expression or under-expression of one or more genes. For example, when a given allele or gene that codes for a protein (or other peptidic molecule) is not being expressed or is not being sufficiently expressed, a formulation as provided herein may be administered to deliver a nucleic acid containing the genetic sequence for the protein of interest to the nucleus of a cell.

In such an instance, the nucleic acid may be a vector that encodes a suitable expression cassette. The expression vector includes all the elements necessary for expressing the gene coding for the protein of interest within the targeted cell so as to produce the encoded protein. The expression cassette is thus capable of directing expression of the nucleic acid within the cell and may therefore code for the regulatory domains required for directing transcription in a host cell. The expression cassette may be designed to preferentially express the encoded protein in a particular host cell type, e.g., the expression cassette may include tissue-specific regulatory elements necessary to express the nucleic acid in a particular cell. A suitable expression cassette, containing the appropriate sequences to control replication in a host cell, may be transformed or otherwise be incorporated into a suitable vector such as those found in bovine papilloma virus type-vectors. Saveria-Campo, M., Bovine Papilloma Virus DNA a Eukaryotic Cloning Vector in DNA Cloning Vol. II a Practical Approach, D. M. Glover, Ed., IRL Press, Arlington, Va. pp. 213-238 (1985).

Accordingly, the expression cassette may include a suitable promoter sequence that is operably linked to a gene encoding the protein or other peptidic molecule of interest. Additionally, the expression cassette may include a polynucleotide sequence encoding one or more of another polypeptide or stable RNA sequence, a regulatory domain, an enhancer, a sequence for accurate splicing of a transcript, a terminator, a selectable marker, or the like.

Tissue-specific and cell type-specific regulatory elements are regulatory elements that are capable of driving transcription in one type of tissue or cell but not in others. Such regulatory elements are known in the art, and include tissue-specific and cell type-specific promoters and enhancers. Non-limiting examples of suitable tissue-specific and/or cell type-specific promoters include the albumin promoter (e.g., liver-specific albumin promoter; see Pinkert et al. (1987) Genes Dev 1:268-277); lymphoid-specific promoters (Calame and Eaton (1988) Adv Immunol 43:235-275), such as promoters of T cell receptors (Winoto and Baltimore (1989) EMBO J 8:729-733) and immunoglobulins (Banerji et al. (1983) Cell 33:729-740; Queen and Baltimore (1983) Cell 33:741-748); neuron-specific promoters (e.g., the neurofilament promoter; see Byrne and Ruddle (1989) PNAS 86:5473-5477); pancreas-specific promoters (Edlund et al. (1985) Science 230:912-916); mammary gland-specific promoters (e.g., milk whey promoter; see U.S. Pat. No. 4,873,316 and European Application Publication No. 264,166); and developmentally regulated promoters, e.g., the murine hox promoters (Kessel and Gruss (1990) Science 249:374-379) and the alpha-fetoprotein promoter (Campes and Tilghman (1989) Genes Dev 3:537-546). Additional promoters include, without limitation, prokaryotic promoters, eukaryotic promoters, yeast promoters, viral promoters, bacteriophage promoters, a tryptophan promoter, a lactose promoter, and the like. The promoter may be a hybrid promoter, a constitutive promoter, a regulatable promoter, an inducible promoter, a homologous promoter, a heterologous promoter, or the like.

The nucleic acid may include an enhancer sequence that acts to increase the expression of the promoter region. Such enhancers may include the SV40 enhancer region, the 35S enhancer element, and the like. Sequences for accurate splicing of a transcript may also be included. An example of a splicing sequence is the VP1 intron from SV40. Sprague, et al., J. Virol. 45: 773-781 (1983). A termination sequence may also be included, such as the polyadenylation sequence from the bovine growth hormone gene (e.g., a “poly-A” tail).

Additionally, the nucleic acid may comprise a selectable marker, i.e., a reporter gene that, once introduced into a cell, results in a trait that enables selection, e.g., antibiotic resistance. In addition to antibiotic resistance markers, examples of selectable markers include, without limitation, auxotrophic markers, metal resistance markers, and the like. For instance, a selectable marker gene may code for one or more of dihydrofolate reductase, puromycin acetyl transferase, neomycin phosphotransferase, blasticidin S, hygromycin B phosphotransferase, guanine phosphoribosyl transferase, and zeocin resistance protein, and selectable traits include resistance to methotrexate, puromycin, neomycin, G418 (Geneticin®, Invitrogen), blasticidin, hygromycin, mycophenolic acid, zeocin, and antibiotics of the beta-lactam family such as penicillins, cephalosporins, carbapenems, etc.

The nucleic acid may additionally encode a protein or other peptidic molecule that treats an adverse condition. Accordingly, the nucleic acid may contain a nucleotide sequence that codes for a protein such as a transcription factor, an enzyme, an antibody, or the like. Hence, a formulation of the invention may be administered to alleviate problems caused by an enzyme deficiency and/or an immune deficiency.

For instance, the encoded protein may be a transcription factor, e.g., a leucine zipper factor, a helix-loop-helix factor, a helix-loop-helix/leucine zipper factor, an NF-1 factor, an RF-X factor, a zinc-coordinating factor, a helix-turn-helix factor, and a β-scaffold factor with minor groove contracts. Zinc-coordinating factors include zinc finger proteins such as the protein may be a zinc finger protein, particularly one of the zinc finger proteins that have been linked to human development and disease. Such zinc finger proteins include, by way of example, C₂H₂, C₄H₄, and C₆H₆ zinc finger proteins.

The encoded protein may also be an enzyme, e.g., an amylase, catalase, cellulase, hydrolase, isomerase, ligase, lipase, lyase, nuclease, oxidoreductase, protease, reductase, or transferase. Specific examples of such enzymes include ribonucleases such as endoribonucleases and exoribonucleases; cholinesterases such as acetylcholinesterase and pseudocholinesterase; hydrolase enzymes such as esterases, phosphatases, glycosidases, phosphatases, peptidases, phospholipases, and metalloproteinases; and lyases such as cyclases. The encoded protein may also be an antigen, an antibody, or a fragment thereof. Antibodies and antibody fragments include, for instance, IgA, IgD, IgE, IgG, IgM, and fragments thereof. With regard to antibody fragments, the nucleic acid may code for a specific region of an antibody, e.g., the Fab region, the Fc region, the heavy chain or regions thereof, or the light chain or regions thereof. When the encoded is an antibody or fragment thereof, a formulation of the invention may invention be administered to treat one or more of cardiovascular disease, inflammatory disease, auto-immune disease, leukemia (such as acute lymphocytic leukemia, chronic lymphocytic leukemia, acute or chronic myelogenous leukemia, and the like), lymphoma (such as Burkett's, Hodgkin, and Non-Hodgkin Lymphomas), cancer (such as colorectal or breast cancer), transplant rejection, multiple sclerosis, Alzheimer's disease, Parkinson's disease, and the like.

Alternatively, the formulation of the invention may include a nucleic acid that is not part of a vector or an expression cassette. For instance, a ribonucleic acid such as an mRNA, a tRNA, an rRNA, or a dsRNA, may be incorporated into a formulation of the invention and delivered to one or more tissues of the body wherein the RNA is not part of a vector or expression cassette. Deoxyribonucleic acids such as an ssDNA, a dsDNA, and/or a tsDNA, may also be incorporated into a formulation of the invention and delivered to one or more tissues of the body wherein the DNA is not part of a vector or expression cassette.

When the adverse condition is caused by the over-expression of a given gene, a formulation, as disclosed herein, is administered so as to deliver to a cell over-expressing the protein a nucleic acid comprising an interfering RNA (iRNA), a micro-interfering RNA (miRNA), a small-interfering RNA (siRNA), and/or an anti-sense RNA, so as to interfere with and/or inhibit transcription or translation of the protein within the cell and thereby alleviate the adverse condition.

That is, RNA molecules such as iRNA and siRNA, are involved in a RNA interference (RNAi) pathway in which these nucleic acids interfere with the expression of specific genes; in the therapeutic context, these genes code for proteins that are being over expressed in a given cell. Specifically, in certain instances, the small interfering RNAs invention delivered using a formulation of the invention may form double-stranded RNA fragments that when administered to a target cell trigger catalytically mediated gene silencing, for example, by targeting an RNA-induced silencing complex (RISC) to bind to and degrade target mRNA, thus effectively silencing a gene of interest. In this manner, the formulations including nucleic acids such as iRNAs have the ability to inactivate essentially any gene of interest, resulting in a therapeutic benefit. Adverse conditions that may be alleviated in this manner include, by way of example, age-related macular degeneration (“AMD”) and Huntington's disease.

The nucleic acid may also be an antisense RNA (aRNA), i.e., a single-stranded RNA molecule that is complementary to another nucleic acid strand, such as a messenger RNA strand that has been transcribed within a cell. Accordingly, a formulation of the invention that includes an aRNA may be administered such that the aRNA is introduced into a cell of interest to inhibit translation of a complementary mRNA by base pairing to it and physically obstructing the translation process.

The biologically active agent may also be a peptidic molecule such as a protein, e.g., invention, a transcription factor, an enzyme, or an antibody. By a “peptidic” molecule, as the term is used herein, is meant any structure comprised of two or more amino acids. For the most part, the peptidic molecules useful in conjunction with the present invention contain about 5 to about 10,000 amino acids. The amino acids forming all or a part of a peptide may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (O), arginine (R), serine (S), threonine (T), valine (V), tryptophan (W), and tyrosine (Y). Any of the amino acids in the peptidic molecules employed herein may be replaced by a non-conventional amino acid. In general, conservative replacements are preferred. Conservative replacements substitute the original amino acid with a non-conventional amino acid that resembles the original in one or more of its characteristic properties (e.g., charge, hydrophobicity, steric bulk; for example, one may replace Val with N-Val). The term “non-conventional amino acid” refers to amino acids other than conventional amino acids, and include, for example, isomers and modifications of the conventional amino acids (e.g., D-amino acids), non-protein amino acids, post-translationally modified amino acids, enzymatically modified amino acids, constructs or structures designed to mimic amino acids (e.g., alpha,alpha-disubstituted amino acids, N-alkyl amino acids, lactic acid, beta-alanine, naphthylalanine, 3-pyridylalanine, 4-hydroxyproline, O-phosphoserine, N-acetylserine, N-formylmethionine, 3-methylhistidine, 5-hydroxylysine, and nor-leucine), and peptides having the naturally occurring amide —CONH— linkage replaced at one or more sites within the peptide backbone with a non-conventional linkage such as N-substituted amide, ester, thioamide, retropeptide (—NHCO—), retrothioamide (—NHCS—), sulfonamido (—SO2NH—), and/or peptoid (N-substituted glycine) linkages. Accordingly, peptidic molecules include pseudopeptides and peptidomimetics. The peptides incorporated into the present formulations can be (a) naturally occurring, (b) produced by chemical synthesis, (c) produced by recombinant DNA technology, (d) produced by biochemical or enzymatic fragmentation of larger molecules, (e) produced by methods resulting from a combination of methods (a) through (d) listed above, or (f) produced by any other means for producing peptides. Peptidic agents herein also include chemically or naturally modified peptidic agents, e.g., glycoproteins and the like.

Peptidic compounds include any pharmacologically active peptide, polypeptide or protein, such as, but not limited to, enzymes, monoclonal and polyclonal antibodies, antigens, coagulation modulators, cytokines, endorphins, peptidic hormones, kinins, and structurally similar bioactive equivalents thereof. By a “structurally similar bioactive equivalent” is meant a peptidic compound with structure sufficiently similar to that of an identified bioactive peptidic compound to produce substantially equivalent therapeutic effects. As used herein and in the appended claims, the terms “protein”, “peptide” and “polypeptide” refer to both the specific peptidic compound(s) identified as well as structurally similar bioactive equivalents thereof.

Peptidic compounds incorporated into the present formulations can be any of those described above with respect to proteins and other peptidic compound coded for by a nucleic acid incorporated into the formulations, i.e., transcription factors, enzymes, antigens, antibodies, antibody fragments, and the like. Specific enzymes of interest include super oxide dismutase (SOD), tissue plasminogen activator (TPA), renin, adenosine deaminase, beta-glucocerebrosidase, asparaginase, dornase-alpha, hyaluronidase, elastase, trypsin, thymidin kinase (TK), tryptophan hydroxylase, urokinase, and kallikrein;

Other peptidic compounds that can be advantageously employed in the present formulations and methods include, but are not limited to, the following:

Coagulation modulators, such as α1-antitrypsin, α2-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, thrombomodulin and combinations thereof;

Cytokines, such as transforming growth factors (TGFs), including TGF-β2, TGF-β2, and TGF-β3; bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), heparin-binding neurotrophic factor (HBNF), and insulin-like growth factor (IGF)); connective tissue activated peptides (CTAPs), osteogenic factors; colony stimulating factor; interferons, including interferon-alpha, interferon alpha-2a, interferon alpha-2b, interferon alpha-n3, interferon-beta, and interferon-gamma; interleukins, including interleukin-1, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, and interleukin-17; tumor necrosis factor; tumor necrosis factor-alpha; granulocyte colony-stimulating factor (G-CSF); granulocyte-macrophage colony-stimulating factor (GM-CSF); macrophage colony-stimulating factor; inhibins (e.g., inhibin A and inhibin B); growth differentiating factors (e.g., GDF-1); activins (e.g., activin A, activin B, and activin AB); midkine (MD); and thymopoietin;

Endorphins, i.e., peptides that activate opiate receptors, including pharmacologically active endorphin derivatives such as dermorphin, dynorphin, alpha-endorphin, beta-endorphin, gamma-endorphin, sigma-endorphin [Leu5]enkephalin, [Met5]enkephalin, substance P, and combinations thereof;

Peptidic hormones, such as activin, amylin, angiotensin, atrial natriuretic peptide (ANP), calcitonin (derived from chicken, eel, human, pig, rat, salmon, etc.), calcitonin gene-related peptide, calcitonin N-terminal flanking peptide, cholecystokinin (CCK), ciliary neurotrophic factor (CNTF), corticotropin (adrenocorticotropin hormone, ACTH), corticotropin-releasing factor (CRF or CRH), follicle-stimulating hormone (FSH), gastrin, gastrin inhibitory peptide (GIP), gastrin-releasing peptide, glucagon, gonadotropin-releasing factor (GnRF or GNRH), growth hormone releasing factor (GRF, GRH), human chorionic gonadotropin (hCH), inhibin A, inhibin B, insulin (derived from beef, human, pig, etc.), leptin, lipotropin (LPH), luteinizing hormone (LH), luteinizing hormone-releasing hormone (LHRH), lypressin, alpha-melanocyte-stimulating hormone, beta-melanocyte-stimulating hormone, gamma-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatostatin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, triiodothyronine, vasoactive intestinal peptide (VIP), and vasopressin (antidiuretic hormone, ADH);

Analogues of LHRH, such as buserelin, deslorelin, fertirelin, goserelin, histrelin, leuprolide (leuprorelin), lutrelin, nafarelin, tryptorelin and combinations thereof;

Kinins, such as bradykinin, potentiator B, bradykinin potentiator C, and kallidin and combinations thereof;

Enzyme inhibitors, such as leupeptin, chymostatin, pepstatin, renin inhibitors, angiotensin converting enzyme (ACE) inhibitors, and the like;

Other peptidic drugs, such as abarelix, anakinra, ancestim, bivalirudin, bleomycin, bombesin, desmopressin acetate, des-Q14-ghrelin, enterostatin, erythropoietin, exendin-4, filgrastim, gonadorelin, insulinotropin, lepirudin, magainin I, magainin II, nerve growth factor, pentigetide, thrombopoietin, thymosin alpha-1, urotensin II, and combinations thereof.

The peptidic agent may be associated with, e.g., conjugated or fused to, one or more of an amino acid sequence comprising a nuclear localization signal (NLS), a cell-penetrating peptide (CPP) sequence, a transactivator domain, such as the VP-16 transactivator, and the like. In this manner, a formulation of the invention including a transport enhancer and a peptidic agent such as a transcription factor or an enzyme, may be administered to a subject so as to deliver a transcription factor or an enzyme of interest. In this manner, a formulation of the invention may be used to treat a condition associated with the under expression of a protein, such as an enzyme.

For instance, a formulation of the invention may include a penetration enhancing agent, such as MSM, and a protein, such as a transcription factor, for the delivery of the transcription factor to a cell and/or through the cell membrane and into the nucleus of the cell, wherein the transcription factor may then faction to up-regulate transcription of a gene of interest, which therefore results in an increase in production of a protein of interest. As indicated above, the transcription factor may be delivered by itself or as a fusion with one or more of an NLS, CPP, and/or transactivator. See, for instance, Tachikawa, K. et al. Regulation of Endogenous VEGF-A gene by exogenous designed regulatory proteins. PNAS (2004) vol. 101, no. 42:15225-15230.

Accordingly, in certain instances, a formulation of the invention may include a transcription factor designed to bind to a DNA binding domain, e.g., promoter region of an endogenous gene, such as a gene encoding a growth factor, hormone (such as an amine derived hormone, a peptide hormone, and a lipid or phospholipid hormone), cytokine, interleukin, and the like. For instance, the formulation may include a transcription factor designed to bind to a promoter region of a gene that encodes or is operably linked to a gene that encodes an acidic fibroblast growth factor (aFGF or FGF-1), basic fibroblast growth factor (bFGF or FGF-2), catecholamine, epidermal growth factor (EGF), erythropoietin (EPO), follicle stimulating hormone, granulocyte-colony stimulating factor (G-CSF) granulocyte-macrophage colony stimulating factor (GM-CSF), growth differentiation factor-9 (GDF9), heparin (e.g., low molecular weight heparin, very low molecular weight heparin, ultra low molecular weight heparin, heparinoids), hepatocyte growth factor (HGF), insulin (e.g., porcine insulin, bovine insulin, human insulin, or human recombinant insulin), interferons (such as IFN-α, β, and γ), interleukins (such as IL-1, IL-2, IL-3, IL-4, IL-5, IL-6-IL-10-IL-13-IL-17, IL-18-IL-35), luteinizing hormone, myostatin (GDF-8), nerve growth factor (NGF), a neurotrophin, platelet-derived growth factor (PDGF), steroid hormones (e.g., testosterone and cortisol), thrombopoietin (TPO), thyroid stimulating hormone, thyroxine, transforming growth factor beta (TGF-α or β), tumor necrosis factor (TNF-α or β), vasopressin, vascular endothelial growth factor (VEGF), and the like.

Accordingly, a formulation of the invention may be administered to ameliorate conditions caused by a reduction in the production of one or more of a growth factor, hormone, cytokine, interleukin, or the like. For instance, a formulation of the invention including a protein such as a growth factor, hormone, cytokine or interleukin, may be administered to treat a condition such as a hematological, oncological, and/or cardiovascular disease, for instance, anaemia, bone marrow diseases, cardiovascular diseases (e.g., the promotion of angiogenesis), leukemias, myelodysplastic syndrome (MDS), neutropenia, transplantation complication, and the like.

It is to be noted that although the protein to be delivered may be a fusion protein including a NLS and/or CPP, in certain instances, the protein does not include an NLS and/or a CPP as the transport enhancer may serve the function of delivering the biologically active agent directly to the cell, and/or through the cell membrane into the cytoplasm of the cell and/or into the nucleus of the cell as desired. For instance, in certain instances, it may be desirable to deliver a biologically active protein to the cell wherein the protein is not conjugated or fused to another molecule. In such an instance, any biologically active protein may be delivered directly in conjunction with the transport enhancer.

When the protein is an antibody, the antibody may be a member selected from the group including an IgA, IgD, IgE, IgG, or IgM antibody or a portion thereof. For instance, the formulation may include a protein fragment or peptide such as an Fab region or an Fc region of an antibody. Specifically, the protein may be an antibody selected from the group including Abciximab, Adalimumab, Alemtuzumab, Basiliximab, Bevacizumab, Cetuximab, Dacilzumab, Eculizumab, Efalizumab, Etanerecept, Gemtuzumab Ozogamicin, Ibritumomab tiuxetan, Infliximab, Muromonab-CD3, Natalizumab, Omalizumab, Palivizumab, Panitumumab, Ranibizumab, Rituximab, Tositumomab, Trastuzumab, and the like, which protein is formulated in conjunction with the transport enhancer to produce a formulation of the invention, which formulation can be administered so as to contact a cell of the body and thereby deliver the protein to the cell.

In certain instances, a suitable biologically active agent to be incorporated in a formulation of the invention may be a biologically active macromolecule such as a hormone. For example, in certain instances, the biologically active agent is a hormone such as a growth hormone; growth hormone-releasing hormone; cytokine; a chemokine; an interferon; an interleukin, such as interleukin-1, interleukin-2, etc., TGF-α, TGF-β; CSF; insulin (e.g., porcine insulin, bovine insulin, human insulin, or human recombinant insulin); insulin-like growth factor (IGF); VEGF; heparin (e.g., low molecular weight heparin, very low molecular weight heparin, ultra low molecular weight heparin, heparinoids); calcitonin; erythropoietin (EPO); atrial naturetic factor; somatostatin; protease inhibitors; adrenocorticotropin; gonadotropin releasing hormone; oxytocin; luteinizing-hormone-releasing-hormone; follicle stimulating hormone; glucocerebrosidase; thrombopoietin; filgrastim; prostaglandins; cyclosporine; vasopressin; cromolyn sodium; vancomycin; desferrioxamine (DFO); parathyroid hormone (PTH); and the like.

In certain instances, a suitable biologically active agent to be incorporated in a formulation of the invention may be a biologically active macromolecule such as one or more of a growth factor, hormone (such as an amine derived hormone, a peptide hormone, and a lipid or phospholipid hormone), cytokine, interleukin, and the like. For instance, the formulation may include a biologically active agent selected from the group including a fibroblast growth factor (aFGF or FGF-1), basic fibroblast growth factor (bFGF or FGF-2), catecholamine, epidermal growth factor (EGF), erythropoietin (EPO), follicle stimulating hormone, gonadotropin releasing hormone, a growth hormone, growth hormone-releasing hormone, granulocyte-colony stimulating factor (G-CSF) granulocyte-macrophage colony stimulating factor (GM-CSF), growth differentiation factor-9 (GDF9), hepatocyte growth factor (HGF), heparin, insulin (e.g., porcine insulin, bovine insulin, human insulin, or human recombinant insulin), insulin-like growth factor (IGF), interferons (such as IFN-α, β, and γ), interleukins (such as IL-1, IL-2, IL-3, IL-4, IL-5, IL-6-IL-10-IL-13-IL-17, IL-18-IL-35), luteinizing hormone, luteinizing-hormone-releasing-hormone, myostatin (GDF-8), nerve growth factor (NGF), a neurotrophin, parathyroid hormone (PTH), platelet-derived growth factor (PDGF), steroid hormones (e.g., testosterone and cortisol), thrombopoietin (TPO), thyroid stimulating hormone, thyroxine, transforming growth factor beta (TGF-α or β), tumor necrosis factor (TNF-α or β), vasopressin, vascular endothelial growth factor (VEGF), and the like.

In certain instances, a suitable biologically active agent to be incorporated in a formulation of the invention may be a biologically active macromolecule such as one or more of the following: heparin, somatostatin, protease inhibitors, adrenocorticotropin, oxytocin, glucocerebrosidase, thrombopoietin, filgrastim, a prostaglandin, cyclosporin, cromolyn sodium, vancomycin, desferrioxamine (DFO), an antigen, an antimicrobial, an anti-fungal agent, a mimetic, alpha-1 antitrypsin, angiostatin, antihemolytic factor, apolipoprotein, apoprotein, atrial natriuretic factor, atrial natriuretic polypeptide, atrial peptide, C—X—C chemokine, T39765, NAP-2, ENA-78, gro-a, gro-b, gro-c, IP-10, GCP-2, NAP-4, SDF-I, PF4, MTG, calcitonin, c-kit ligand, cytokine, CC chemokine, monocyte chemoattractant protein-1, monocyte chemoattractant protein-2, monocyte chemoattractant protein-3, monocyte inflammatory protein-1 alpha, monocyte inflammatory protein-1beta, RANTES, 1309, R83915, R91733, HCC1, T58847, D31065, T64262, CD40, CD40 ligand, c-kit ligand, collagen, complement factor 5a, complement inhibitor, complement receptor 1, epithelial neutrophil activating peptide-78, MIP-16, MCP-1, epidermal growth factor (EGF), epithelial neutrophil activating peptide, exfoliating toxin, Factor IX, Factor VII, Factor VIII, Factor X, fibroblast growth factor (FGF), fibrinogen, fibronectin, four-helical bundle protein, glp-1, glucocerebrosidase, gonadotropin, growth factor receptor, growth-hormone-releasing hormone (GRF or GHRF; also known as growth-hormone releasing hormone, or GHRH), hedgehog protein, hemoglobin, hepatocyte growth factor (hGF), hirudin, human growth hormone (hGH), human serum albumin, ICAM-1, ICAM-1 receptor, LFA-I, LFA-I receptor, insulin, insulin-like growth factor (IGF), IGF-I, IGF-II, interferon OFN), IFN-alpha, IFN-beta, TFN-gamma, keratinocyte growth factor (KGF), lactoferrin, leukemia inhibitory factor, luciferase, neurturin, neutrophil inhibitory factor (NIF), oncostatin M, osteogenic protein, oncogene product, paracitonin, parathyroid hormone (PTH), PD-ECSF, platelet-derived growth factor (PDGF), peptide hormone, pleiotropin, protein A, protein G, pyrogenic exotoxin A, pyrogenic exotoxin B, pyrogenic exotoxin C, Peptide YY (PYY), relaxin, renin, stem cell factor (SCF), SCF-complex, small biosynthetic protein, soluble complement receptor I, soluble I-CAM 1, soluble interleukin receptor, soluble TNF receptor, somatomedin, somatostatin, somatotropin, streptokinase, superantigens, staphylococcal enterotoxin, SEA, SEB, SEC1, SEC2, SEC3, SED, SEE, steroid hormone receptor, superoxide dismutase, toxic shock syndrome toxin, thymosin alpha 1, tissue plasminogen activator, tumor growth factor (TGF), tumor necrosis factor receptor (TNFR), VLA-4 protein, VCAM-1 protein, vascular endothelial growth factor (VEGF), urokinase, mos, ras, raf, met, p53, tat, fos, myc, jun, myb, rel, estrogen receptor, progesterone receptor, testosterone receptor, aldosterone receptor, LDL receptor, corticosterone, an anti-microbial agent, an anti-fungal agent, a polysaccharide, a lipopolysaccharide, and/or a lipid, and polyethylene glycol (PEG)-modified derivatives of these compounds; and any combination thereof.

The biologically active agent may also be lipidic. Lipidic agents include, for example, lipopolysaccharides (LPS; also referred to as lipoglycans); fatty acids and conjugates and derivatives thereof, including fatty esters, fatty amides, fatty alcohols, and eicosanoids such as prostaglandins and thromboxanes; glycerophospholipids such as phosphatidylcholine, phosphatidylethanolamine, and phosphatidylserine; sphingolipids such as sphingomyelins, cerebrosides, and gangliosides; and polyketides.

Saccharide-based active agents include disaccharides, oligosaccharides, polysaccharides, mucopolysaccharides, peptidoglycans (peptido-polysaccharides), exopolysaccharides, and the like.

In certain instances, the biologically active agent to be incorporated in a formulation of the invention may be a biologically active small molecule or a combination of a small molecule (such as one or more of a non-steroidal anti-inflammatory agent, steroidal anti-inflammatory agent, a corticosteroid, or the like, as described below) and a macromolecule, as set forth above.

For instance, the biologically active agent may be combined with or may be a non-steroidal anti-inflammatory drug (NSAID). Suitable NSAIDs that may be used in the formulations of the present invention include, but are not limited to: propionic acid derivatives such as ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, fenbufen and tiaprofenic acid; acetylsalicylic acid; apazone; diclofenac; difenpiramide; diflunisal; etodolac; flufenamic acid; indomethacin; ketorolac; meclofenamate; mefenamic acid; oxicams such as meloxicam and piroxicam; nabumetone; phenylbutazone; piroxicam; salicylates such as salsalate and acetylsalicylic acid; sulfasalazine; sulindac; tolmetin; Cox-2 inhibitors such as celecoxib, rofecoxib, and valdecoxib, and combinations of any of the foregoing. Other biologically active agents include steroidal anti-inflammatory agents based on the use of corticosteroids and leukotrienes. These include, but are not limited to, orally and parenterally administered corticosteroids, inhaled corticosteroids, and leukotriene modifiers (e.g., montelukast, zileuton, and zafirlukast).

Suitable examples of orally and/or parenterally administrable corticosteroids include, but are not limited to, cortisone, hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisone, prednisolone, methylprednisolone, clobetasol (e.g., as clobetasol propionate), betamethasone (e.g., as betamethasone benzoate and betamethasone diproprionate), fluocinonide, diflorasone diacetate, mometasone (e.g., as mometasone furoate), triamcinolone (e.g., as triamcinolone acetonide) Corticosteroids that are typically administered via inhalation include beclomethasone, budesonide, mometasone, triamcinolone, flunisolide, and fluticasone. Other anti-inflammatory agents that may serve as biologically active agents herein include carboxyamidotriazole, combretastatin A-4, squalamine, 6-O-chloroacetyl-carbonyl)-fumagillol, thalidomide, angiostatin, troponin-1, angiotensin II antagonists, hydroxychloroquinone, penicillamine, and the like. Other anti-inflammatory agents that can be advantageously employed in the present formulations and methods include, by way of example:

(a) Leukotriene biosynthesis inhibitors, 5-lipoxygenase (5-LO) inhibitors, and 5-lipoxygenase activating protein (FLAP) antagonists, such as zileuton; ABT-761; fenleuton; tepoxalin; Abbott-79175; Abbott-85761; N-(5-substituted)-thiophene-2-alkylsulfonamides; 2,6-di-tert-butylphenol hydrazones; Zeneca ZD-2138; SB-210661; pyridinyl-substituted 2-cyanonaphthalene compound L-739,010; 2-cyanoquinoline compound L-746,530; indole and quinoline compounds MK-591, MK-886; and BAY x 1005;

(b) Receptor antagonists for leukotrienes LTB4, LTC4, LTD4, and LTE4, including phenothiazin-3-one compound L-651,392; amidino compound CGS-25019c; benzoxazolamine compound ontazolast; benzenecarboximidamide compound BIIL 284/260; compounds zafirlukast, ablukast, montelukast, pranlukast, verlukast (MK-679), RG-12525, Ro-245913, iralukast (CGP 45715A), and BAY x 7195;

(c) 5-Lipoxygenase (5-LO) inhibitors; and 5-lipoxygenase activating protein (FLAP) antagonists;

(d) Dual inhibitors of 5-lipoxygenase (5-LO) and antagonists of platelet activating factor (PAF);

(e) Leukotriene antagonists (LTRAs) of LTB4, LTC4, LTD4, and LTE4;

(f) Antihistaminic H1 receptor antagonists, including, cetirizine, loratadine, desloratadine, fexofenadine, astemizole, azelastine, and chlorpheniramine;

(g) Gastroprotective H2 receptor antagonists;

(h) Beta₁- and beta₂-adrenoceptor agonist vasoconstrictor sympathomimetic agents administered orally or topically for decongestant use, including propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride;

(i) one or more beta₁- and beta₂-adrenoceptor agonists as recited in (h) above in combination with one or more inhibitors of 5-lipoxygenase (5-LO) as recited in (a) above;

(j) Theophylline and aminophylline;

(k) Sodium cromoglycate;

(l) Muscarinic receptor (M1, M2, and M3) antagonists;

(m) COX-1 inhibitors (NTHEs); and nitric oxide NTHEs;

(n) COX-3 inhibitor for example acetaminophen;

(o) insulin-like growth factor type I (IGF-1) mimetics;

(p) Ciclesonide;

(q) Tryptase inhibitors;

(r) Platelet activating factor (PAF) antagonists;

(s) Monoclonal antibodies active against endogenous inflammatory entities;

(t) IPL 576;

(u) Anti-tumor necrosis factor (TNF-beta) agents, including etanercept, infliximab, and D2E7;

(v) DMARDs for example leflunomide;

(w) Elastase inhibitors, including UT-77 and ZD-0892;

(x) TCR peptides;

(y) Interleukin converting enzyme (ICE) inhibitors;

(z) IMPDH inhibitors;

(aa) Adhesion molecule inhibitors including VLA-4 antagonists;

(bb) Cathepsins;

(cc) Mitogen activated protein kinase (MAPK) inhibitors;

(dd) Mitogen activated protein kinase kinase (MAPKK) inhibitors;

(ee) Glucose-6 phosphate dehydrogenase inhibitors;

(ff) Kinin-B1- and B2-receptor antagonists;

(ii) Gold in the form of an aurothio group in combination with hydrophilic groups;

(jj) Immunosuppressive agents, including cyclosporine, azathioprine, tacrolimus, and methotrexate;

(gg) Anti-gout agents, including colchicine;

(hh) Xanthine oxidase inhibitors, including allopurinol;

(ii) Uricosuric agents, including probenecid, sulfinpyrazone, and benzbromarone;

(jj) Antineoplastic agents that are antimitotic drugs for example vinblastine, vincristine, cyclophosphamide, and hydroxyurea;

(kk) Growth hormone secretagogues;

(ll) Inhibitors of matrix metalloproteinases (MMPs), including the stromelysins, the collagenases, the gelatinases, aggrecanase, collagenase-1 (MMP-1), collagenase-2 (MMP-8), collagenase-3 (MMP-13), stromelysin-1 (MMP-3), stromelysin-2 (MMP-10), and stromelysin-3 (MMP-11);

(mm) Transforming growth factor (TGF-α or β);

(nn) Platelet-derived growth factor (PDGF);

(oo) Fibroblast growth factor, including basic fibroblast growth factor (bFGF);

(pp) Granulocyte macrophage colony stimulating factor (GM-CSF);

(qq) Capsaicin;

(rr) Tachykinin NK1 and NK3 receptor antagonists, including NKP-608C; SB-233412 (talnetant); and D-4418; and

(ss) A2A receptor agonist, or any combinations thereof

In addition to such pharmaceutical agents, a formulation of the invention may also include an herbal species that has anti-inflammatory properties, such as hyssop, ginger, Arnica montana (which contains helenalin), a sesquiterpene lactone, and willow bark, which contains salicylic acid, a substance related to the active ingredient in aspirin. These herbs are encompassed by the present invention and one or more herbs can be combined in a formulation with one or more transport enhancers.

Additionally, other suitable small molecule biologically active agents include one or more of the following as an active agent: an antioxidant, vitamin A, vitamin C, vitamin E, lycopene, selenium, alpha-lipoic acid, coenzyme Q, glutathione, a carotenoid, a metal complexer, a chelator, such as (EDTA), an antibiotic, and an antihistamine. Other suitable small molecule biologically active agents include one or more of the following as an active agent: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isofluorophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinolone, trifluoromethazolamide, trifluridine, trimethoprim, tropicamide, unoprostone, vidarabine, xylometazoline, pharmaceutically acceptable salts thereof, and combinations thereof

The concentrations of the transport enhancer and biologically active agent in the formulation are also of interest. In general, enhancer concentrations on the order of a few percent by weight may be used in liquid formulations for parenteral administration, for example in the range of about 0.01 wt. % or less to about 10 wt. % or more, typically in the range of about 0.1 wt. % to about 10 wt. %, more typically in the range of about 1 wt. % to about 6 wt. %, and most typically in the range of about 2 wt. % to about 4 wt. %, for instance, 3 wt. %. In liquid formulations for parenteral administration, the concentration of biologically active agent will also, generally, be within the aforementioned ranges. A representative such formulation, with MSM as the transport enhancer and a macromolecule as the active agent, contains about 5 wt. % MSM and in the range of about 0.01 wt. % to about 2 wt. % biologically active agent.

Without being bound by theory, it is believed that the transport enhancer in formulations of the invention assists in the process of transporting the biologically active agent not just into the tissue, but across biological membranes and to the site at which the active agent is needed. For instance, the transport enhancer and biologically active agent may, in certain instances, form a stable moiety that is capable of migrating to a target site where the biologically active agent interacts with a cell surface moiety (e.g., a receptor or ion channel), an intracellular molecule (such as an enzyme or other substrate), or a nuclear molecule (such as a DNA, RNA, or nuclear protein), thereby treating an adverse condition.

Methods of Use and Adverse Conditions to be Treated:

It has been determined that many of the current existing transport systems known in the art are limited for use in general therapeutic application, for instance, because they are either inefficient, have the potential for adversely affecting the host genome, may alter the biological properties of the active agent, may kill the target cell, or may pose a risk if used in a human subject, such as when viral conjugates are used as delivery vehicles.

Therefore, as described above, in one aspect, the subject invention provides methods of using a formulation, which includes a transport enhancer and a biologically active agent, to translocate an agent across a biological membrane of a cell, which formulation overcomes the deficiencies in the prior art. In certain instances, the translocation is for experimental and/or diagnostic purposes, and, in certain instances, the translocation is for the treatment of an adverse biological condition.

Accordingly, in one embodiment, the invention provides a method of transporting a biologically active agent (which may also be a test agent) into a cell. The agent is to be contacted with a cell. The cell may be provided in vitro, in vivo, ex vivo, or the like. Accordingly, the cell may be one that has been removed from a tissue and/or from the body, may be present within a tissue (such as within an organ) of the body (which organ or tissue may be present within the body or removed therefrom), or the cell may be one that has been removed from the body with the expectation that the cell is to be returned to the body, for instance, a gamete cell. The method further includes contacting the cell with a transport enhancer.

Hence, a formulation of the present invention uses a transport enhancer, such as methylsulfonylmethane, for translocating agents, such as biologically active or test agents, across cellular membranes, for the delivery, e.g., intracellular delivery, of beneficial agent to a cell. Hence, the invention also provides a method of introducing a biologically active agent of interest into a cell or a cell nucleus. The method includes contacting the cell with a formulation including a transport enhancer and a biologically active agent, in an amount sufficient to enable efficient penetration into the cells. As stated above, in general, the method may be used for in vivo or in vitro internalization of the biologically active agent. For example, the formulation including the biologically active agent may be provided in vitro, ex vivo, or in vivo.

Furthermore, in certain instances, the transport enhancer, such as MSM, is capable of increasing the biological activity or otherwise increasing the efficacy of the active agent. Therefore, a method for using a transport enhancer that increases the biological activity of the agent with which it is associated is also provided. According to the in vitro method, a transport enhancer may be associated with a biologically active agent in a suitable biologically acceptable carrier, and the formulation may be administered, incubated, or otherwise contacted with cells under conditions that enable the active agent to penetrate into and through the cell surface membrane and/or into the nucleus of the cell.

Therefore, in one embodiment, the invention provides a method for increasing the cellular concentration of biologically active agent within a target cell, such as a prokaryotic or eukaryotic cell (e.g., a human cell), whereby the biologically active agent is contacted with the cell in conjunction with a suitable transport enhancer, as described herein, under conditions promoting active metabolism of the cell. In another embodiment, the method provides for translocating a biologically active agent into the cytoplasm and/or nucleus of a eukaryotic cell, whereby the biologically active agent is introduced into the cell by being incubated along with a suitable transport enhancer in a cell culture with the cell.

The target cell, tissue, organ, etc. may be contacted with the active agent and transport enhancer (e.g., methylsulfonylmethane) in any logical order. For instance, the cell may be contacted first with the transport enhancer prior to the cell being contacted with the biologically active/test agent or vice-versa. In certain instances, the transport enhancer and the biologically active agent are mixed to form a composition mixture prior to contacting the cell with the composition mixture.

In some embodiments, the present invention provides a method of ameliorating an adverse condition in a subject, comprising: administering to a subject suspected to be in need thereof a formulation comprising methylsulfonylmethane, a macromolecule, and a pharmaceutically acceptable carrier. In certain instances, the macromolecule may include a nucleic acid, a protein, a polysaccharide, a lipopolysaccharide, a lipid, and the like.

For instance, it has been observed that a formulation of the invention including a transport enhancer, such as methylsulfonylmethane, is effective for translocating agents, such as biologically active or test agents, across cellular membranes, both in vivo and in vitro, especially when the cell is contacted with a formulation of the invention under conditions sufficient to effect transport of the active agent into the cell.

The present formulations overcome the limitations of prior transport systems insofar as transport of a biologically active agent, as disclosed herein, is more rapid and efficient in the presence of a transport enhancer such as methylsulfonylmethane, than in the absence of the transport. The present formulations, therefore, exhibit efficient delivery of a biologically active agent that does not affect the biological agent and is otherwise non-invasive.

Accordingly, in certain instances, a method for transporting a biologically active or test agent into a cell is provided. The method may include contacting a cell with a transport enhancer, e.g., methylsulfonylmethane, and a biologically active agent or test agent, such as a macromolecule, under conditions sufficient to effect transport of the agent into the cell. In certain instances, the present invention provides a transport system that may target various cell types for the intracellular delivery of a biologically active agent to the surface, cytoplasm, and/or nucleus of the cell.

Hence, in certain embodiments, a method for administering a biologically active agent to a mammal, such as a human or an animal, is provided. The method involves administering a formulation to the subject that contains a transport enhancer as described herein, a biologically active agent, and a biologically acceptable carrier. Accordingly, in certain embodiments, the invention provides a pharmaceutical formulation containing a transport enhancer and a therapeutically effective amount of a biologically active agent, and a biologically or pharmaceutically acceptable carrier. A formulation of the invention may be administered to a subject for the treatment of an adverse condition in an amount sufficient to treat the condition. For example, the formulation may be administered so as to treat an adverse condition such as diabetes, cancer (such as colon cancer), liver disease, kidney disease, anemia, respiratory ailments, neurodegenerative disorders, cardioplegia, viral infections, or any of the other conditions disclosed herein, or the like.

With regard to the treatment of diabetes, it should be noted, at the outset, that β-cell mass is typically tightly regulated so that insulin secretion maintains normoglycemia. Fitting β-cell mass to the needs of an infant or the adult organism, particularly when faced with physiological and physiopathological obstacles, is essentially attained by a dynamic balance between β-cell death and β-cell regeneration that occurs from differentiation of immature β-cells and from the proliferation of preexisting insulin-secreting cells.

In Type I diabetes, impaired balance results from accelerated β-cell destruction, a process initiated by a specific attack of a subject's immune system that targets pancreatic sells. Preventing or decreasing the rate of β-cell destruction may therefore not only help stabilize diabetes, but may also allow for islet regeneration to correct β-cell mass insufficiency.

Several molecules are known to be important in decreasing the rate of β-cell loss in experimental models of Type I diabetes. Many of these molecules are peptidic in nature and may be incorporated into formulations of the invention. These peptidic compounds, as described in greater detail infra, serve as basis for the design of pharmaceutical formulations of the invention, namely including a transport enhancer and a biologically active agent, a biologically acceptable carrier, and/or one or more effector agents.

Accordingly, in one embodiment, a formulation of the present invention targets β-cell intracellular mechanisms for the treatment of Type I diabetes. Type I diabetes is secondary to the destruction of the pancreatic β-cells by secretion of the immune system. Data, both in human and rodents, indicate that the cytokines interleukin-1β(IL-1β), in conjunction with TNFα and IFNγ, secreted by macrophages and T-cells, are components responsible for the outcome that leads to β-cell dysfunction and destruction that results in Type I diabetes.

These secreted cytokines engage in a highly complex network of signaling and effector molecules in pancreatic β-cells. The signaling modifies the comportment of the cells and has an impact on the cell fate. This regulatory intracellular network represents a good target for the development of a novel therapeutic approach, as described herein. Each of the molecules involved in the intracellular cytokine signaling pathway represents a target for the formulations of the present invention.

For instance, macromolecules, such as nucleic acids and proteins that target and inhibit the transcription, translation, production, and/or functioning of these cytokines may be delivered in a formulation of the invention so as to prevent their production and/or function, and thereby prevent and/or treat Type 1 diabetes. Specifically, iRNA or aRNA targeted to the genes that encode such cytokines may be included in a formulation of the invention and administered to prevent diabetes. Similar strategies for producing formulations targeted to treat diseases associated with adherent cascades within other pancreatic cells, hepatocytes, colon cells, muscle cells and/or lung cells are also contemplated herein.

For instance, among the many prominent signaling molecules recruited by IL-1β in β-cells are ceramides, prostaglandins, heat-shock proteins, the inducible NO synthase enzyme (iNOS), the transcription factor NF-kappa-B, and the three MAP kinases ERK1/2, p38 and JNK. Many of these molecules are targets for blockage with existing inhibitors that have led to improvement of β-cell survival and function. For example, iNOS KO mice are resistant to IL-1β cytotoxicity and blockers of iNOS activity prevent different aspects of NO cytotoxicity. Islets and cell-lines studies have indicated that blockers of Ca2+ channels or caspase inhibitors prevent rodent β-cell death. p38 inhibitors attenuate IL-1β-mediated inhibition of glucose-stimulated insulin release. B-cell specific suppression of GAD expression in antisense GAD transgenic NOD mice prevented autoimmune diabetes. Expression of bcl-2, IL-1Ra as do JBD (a dominant inhibitor of the c-Jun N-terminal Kinase JNK) in pancreatic β-cell lines had lead to the generation of cells that resist apoptosis. Together, these data indicate that the manipulation of intracellular events with such therapeutic molecules holds great promise for the treatment of Type I diabetes.

Difficulties have persisted in the delivery of these therapeutic agents to the cells of the body. One major challenge has been the difficulty of converting biologically important molecules into bioactive, cell-permeable compounds that are usable in vivo. For example, the most promising tools for the prevention of β-cell loss are a number of large proteins (e.g., Bcl-2, inhibitors of cytokine signaling, such as dominant negative versions of MyD88, TRAF, FADD or IRAK, or the JNK inhibitor JBD280) that are difficult to efficiently deliver in vivo to various tissues and cell types including pancreatic β-cells. The present formulations overcome this difficulty in that these macromolecules can now be efficiently and effectively translocated into cells of the body.

Formulation Types and Modes of Administration

The formulations of the invention comprise a transport enhancing amount (or concentration) of a transport enhancer as described herein, in addition to a therapeutically effective amount of the biologically active agent to be delivered. The formulations also contain a pharmaceutically acceptable carrier adapted to a particular formulation type, e.g., oral, parenteral, or the like. The term “therapeutically effective amount” refers to an amount of the transport enhancer and/or biologically active agent that is nontoxic and capable of achieving a beneficial endpoint or therapeutic effect (e.g., decreasing symptoms associated with inflammation).

A variety of means can be used to formulate the compositions of the invention. Techniques for formulation and administration may be found in “Remington: The Science and Practice of Pharmacy,” Twentieth Edition, Lippincott Williams & Wilkins, Philadelphia, Pa. (1995). For human or animal administration, preparations should meet sterility, pyrogenicity, general safety and purity standards comparable to those required by the FDA. Administration of the pharmaceutical formulation can be performed in a variety of ways, as described herein.

The biologically active agent may be administered, if desired, in the form of a salt, ester, amide, prodrug, active metabolite, isomer, analog, crystalline form, hydrate, or the like, provided that the salt, ester, amide, prodrug, active metabolite, isomer, analog, crystalline form, hydrate, etc is pharmaceutically acceptable. Salts, esters, amides, prodrugs, etc. may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992).

For those biologically active agents that are chiral in nature and can thus be in enantiomerically pure form or in a racemic mixture, the active agent may be incorporated into the present dosage units either as the racemate or in enantiomerically pure form.

The amount of biologically active agent administered will depend on a number of factors and will vary from subject to subject and depend on the particular biologically active agent, the particular disorder or condition being treated, the severity of the symptoms, the subject's age, weight and general condition, and the judgment of the prescribing physician.

The term “dosage form” denotes any form of a pharmaceutical composition that contains an amount of biologically active agent and transport enhancer sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics, such as hydrophilicity.

The formulations of the present invention can also be formulated for controlled release or sustained release. The term “controlled release” refers to a pharmaceutical formulation in which release of the biologically active agent is not immediate, e.g., with a “controlled release” formulation, administration does not result in immediate release of the drug into an absorption pool. The term is used interchangeably with “nonimmediate release” as defined in Remington: The Science and Practice of Pharmacy, cited previously. In general, the term “controlled release” as used herein includes sustained release and delayed release formulations.

The term “sustained release” (synonymous with “extended release”) is used in its conventional sense to refer to a pharmaceutical formulation that provides for gradual release of an active agent over an extended period of time, and which preferably, although not necessarily, results in substantially constant blood levels of a drug over an extended time period.

The present formulations may also include conventional additives such as opacifiers, antioxidants, fragrance, colorant, gelling agents, thickening agents, stabilizers, surfactants, and the like. Other agents may also be added, such as antimicrobial agents, to prevent spoilage upon storage, i.e., to inhibit growth of microbes such as yeasts and molds. Suitable antimicrobial agents are typically selected from the methyl and propyl esters of p-hydroxybenzoic acid (i.e., methyl and propyl paraben), sodium benzoate, sorbic acid, imidurea, and combinations thereof.

Depending on the intended mode of administration, the pharmaceutical formulation may be a solid, semi-solid or liquid, such as, for example, a tablet, a capsule, a caplet, a liquid, a suspension, an emulsion, a suppository, granules, pellets, beads, a powder, or the like, preferably in unit dosage form suitable for single administration of a precise dosage. Suitable pharmaceutical formulations and dosage forms may be prepared using conventional methods known to those in the field of pharmaceutical formulation and described in the pertinent texts and literature, e.g., in Remington: The Science and Practice of Pharmacy, cited previously herein.

The dosage regimen will depend on a number of factors that may readily be determined, such as severity of the condition and responsiveness of the condition to be treated, but will normally be one or more doses per day, with a course of treatment lasting from several days to several months, or until a cure is effected or a diminution of disease state or other adverse condition is achieved.

For orally active biologically active agents, i.e., agents that exhibit sufficient bioavailability to be therapeutically effective when administered orally in a formulation of the invention, oral administration is preferred. Oral dosage forms, as is well known in the art, include tablets, capsules, caplets, solutions, suspensions and syrups, and may also comprise a plurality of granules, beads, powders, or pellets that may or may not be encapsulated. Preferred oral dosage forms are tablets and capsules.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like. Capsules may also be used as an oral dosage form for those compounds that are orally active, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals.

Oral dosage forms, whether tablets, capsules, caplets, or particulates, may, if desired, be formulated so as to provide for gradual, sustained release of the active agent over an extended time period. Generally, as will be appreciated by those of ordinary skill in the art, sustained release oral dosage forms are formulated by dispersing the active agent within a matrix of a gradually hydrolyzable material such as a hydrophilic polymer, or by coating a solid, drug-containing dosage form with such a material.

The compositions of the present disclosure can also be administered parenterally to a subject/patient in need of such treatment. The term “parenteral” generally encompasses any systemic mode of administration other than oral administration.

Preparations for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

The preparation of more, or highly, concentrated solutions for subcutaneous or intramuscular injection is also contemplated. In this regard, the use of DMSO as solvent, in addition to MSM, is preferred as this will result in extremely rapid penetration, delivering high concentrations of the active agent to a small area.

The formulations can also be administered topically to a subject in need of treatment. The term “topical administration” is used in its conventional sense to mean delivery (e.g., process of applying or spreading one or more compositions according to the instant disclosure onto the surface of the skin) to a predetermined area of skin or mucosa of a subject, as in, for example, the treatment of various skin disorders. Topical administration, in contrast to transdermal administration, is intended to provide a local rather than a systemic effect. In certain instances, as may be stated or implied by the circumstances, the terms “topical drug administration” and “transdermal drug administration” may be used interchangeably.

By “predetermined area” of skin or mucosal tissue, which refers to the area of skin or mucosal tissue through which an active agent—transport enhancer formulation is delivered, is intended a defined area of intact unbroken living skin or mucosal tissue, or in certain instances, broken skin, such as skin that includes an abrasion or cut. That area will usually be in the range of about 5 cm² to about 200 cm², more usually in the range of about 5 cm² to about 100 cm², preferably in the range of about 20 cm² to about 60 cm². It will be appreciated, however, that the area of skin or mucosal tissue through which drug is administered may vary significantly, depending on patch configuration, dose, and the like.

Suitable formulations for topical administration include ointments, creams, gels, lotions, pastes, and the like, and may contain irritation-mitigating additives to minimize or eliminate the possibility of skin irritation or skin damage resulting from the pharmacologically active base or other components of the composition. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. Topical formulations may also be prepared with liposomes, micelles, and microspheres.

The compositions of the present disclosure can also be administered nasally to a subject/patient in need of such treatment. The term “nasal” as used herein is intended to encompass delivery through the mucosa of the nasal cavity, throat, and/or lungs. For instance, formulations for nasal administration can be prepared with standard excipients, e.g., as a solution in saline, as a dry powder, or as an aerosol and may be administered by a metered dose inhaler (MDI), dry powder inhaler (DPI) or a nebulizer.

In addition to the aforementioned delivery systems, a composition of the invention may also be formulated as a depot preparation for controlled release of the active agent, preferably sustained release over an extended time period. These sustained release dosage forms are generally administered by implantation (e.g., subcutaneously or by intramuscular injection). Although the present compositions will generally be administered orally, parenterally, topically, transdermally, or via an implanted depot, other modes of administration are suitable as well. For example, administration may be rectal or vaginal, preferably using a suppository that contains, in addition to the active agent, excipients such as a suppository wax. For suppositories, traditional binders and carriers may include, for example, polyalkylene glycols or triglycerides; such suppositories may be formed from mixtures containing the active ingredient in the range of 0.5% to 10%, preferably 1%-2%.

Systemic administration is generally preferred herein, and includes parenteral and enteral administration, resulting in a systemic, i.e., “non-local” effect, as opposed to topical administration, which results in a solely local effect.

### EXAMPLE

The following examples are put forth so as to provide those skilled in the art with a complete invention and description of how to make and use embodiments in accordance with the invention, and are not intended to limit the scope of what the inventors regard as their discovery. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

In accordance with the methods of the invention, human neuroblastoma cells (SH-SY5Y) were purchased from the ATCC Cell Bank. The cells were cultured in 50% Ham's F12 and 50% minimum essential medium supplemented with 10% fetal bovine serum and 2.0 mM L-glutamine at 37 degrees C. in a 5.% CO₂ humidified atmosphere.

An exemplary protocol is as follows: The cells (0.25×106) with 1 ml complete medium were seeded in each well of two four-well chamber slides, incubated at 37 degrees C. for 30 minutes to 24 hours to let the cells adhere to the slides and become about 80% confluent. Assignment: (a) control—untreated cells (100 μl serum-free medium, i.e., “SFM”); (b) with the active agent but without MSM; (c) with the active agent and MSM; (d) with MSM and mixed well and incubated at 37 degrees C. for 60 minutes; and (e) with FuGENE 6 transfection reagent added −80 μl SFM+5 μl 20 mM Cy3-SiRNA (dsRNA)+15 μl transfection reagent, mixed well and incubated at room temperature for 30 minutes.

The solutions (a)-(e) were added into proper wells. Then, 900 μl of complete medium was added to all the chambers and the slides were placed in the 37 degree C. incubator overnight. Syto-16 Green was prepared (1:100, 10 μM)-792 μl of SFM+8 μl of Syto-16 (1.0 mM), mixed well, and added to each well and incubated at 37 degrees C. for 30 minutes. The medium with the dyes was removed from chamber slides. Slides were washed 3× with PBS, and then fixed in 10% paraformaldehyde at room temperature for 10 minutes, followed by another PBS wash. The slides were preserved with mounting medium. The slides were observed under a confocal microscope or a fluorescent light microscope, and photographs captured.

The materials included EDTA (tetrasodium salt) and MSM, which were purchased from Sigma. All cell culture medium components were obtained from Invitrogen. Si-GLO transfection indicator was from Thermo Scientific Dharmcon. SYTO green fluorescent nucleic acid stain (Syto-16) was from Invitrogen. FuGENE 6 transfection reagent was purchased from Roche Applied Science.

### Example I

### dsRNA Delivery

In accordance with the methods of the disclosure, human Neuroblastma cells (SH-SY5Y) were prepared and cultured as indicated above. Specifically, the SH-SY5Y cells (0.25×106) with 1 ml complete medium were seeded in each well of two 4-well chamber slides, incubated at 37° C. for 4 hours to let the cells adhere to slides and to become about 80% confluent. Photographs of the neuroblastoma cells were taken with a confocal microscope and the results are shown below.

Assignment:

a. Control: Untreated cells (100 μl Serum free medium (SFM)),

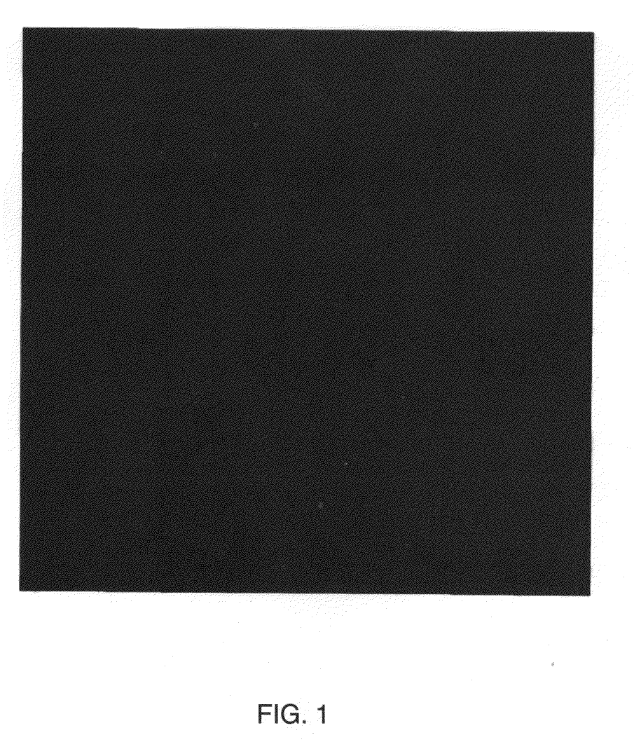

Without MSM, added 100 nM dsRNA-FITC: (95 μl SFM+5 μl 20 mM dsRNA-FITC). The results are shown in FIG. 1. FIG. 1 shows the delivery of a dsRNA-FITC transcript into the neuroblastoma cell where the dsRNA is delivered in the absence of MSM. As can be seen from the sparse spots very little of the dsRNA-FITC has passed through the cell membrane and entered the cell;

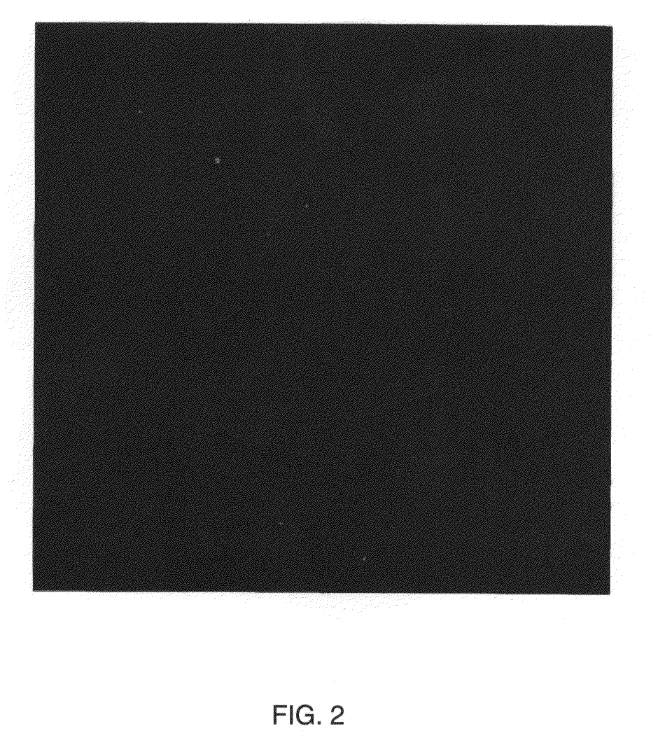

c. With 4 mM MSM, added 100 nM dsRNA-FITC (85 μl SFM+5 μl 20 mM dsRNA-FITC+10 μl 400 mM MSM) mixed well. The results are shown in FIG. 2. FIG. 2 shows the delivery of dsRNA-FITC into the neuroblastoma cell where the dsRNA is delivered in the presence of MSM. As can be seen from the distinct spots, the dsRNA-FITC transcript has passed through a number of cell membranes and entered a number of cells;

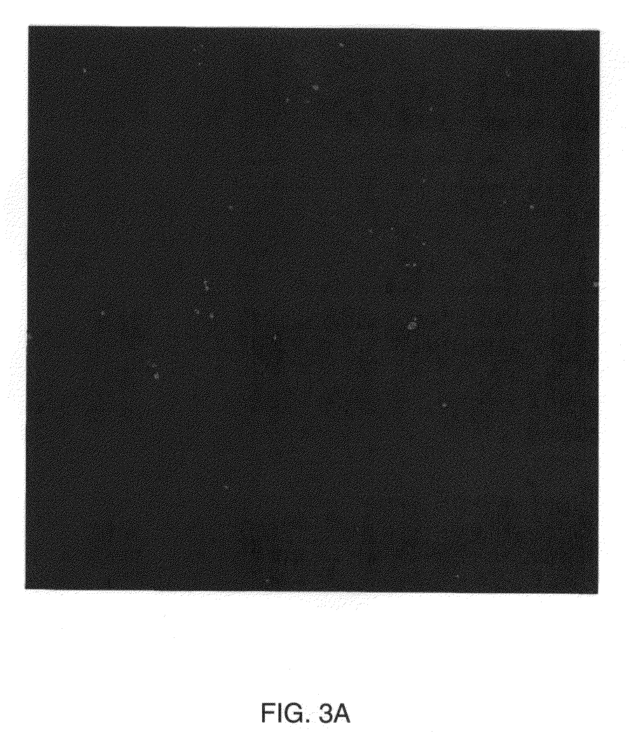

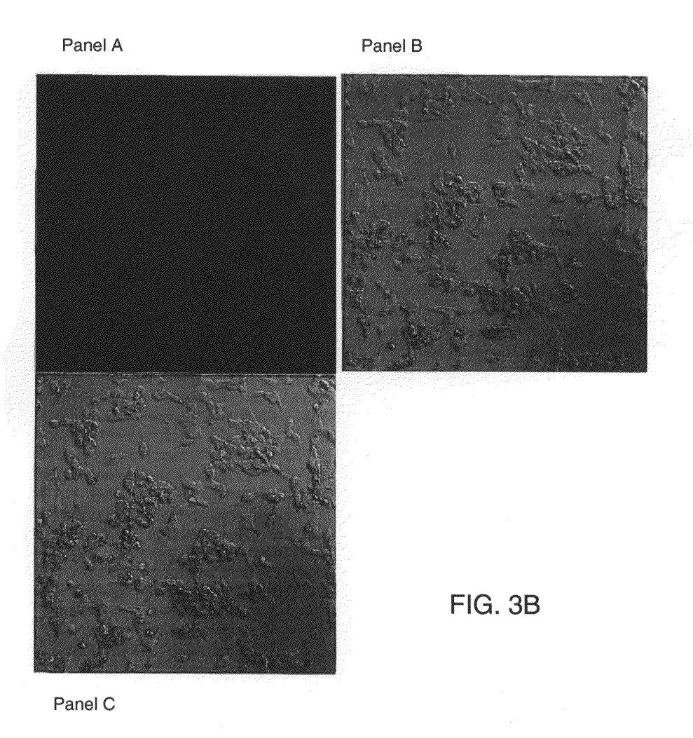

d. With 4 mM MSM, added 100 nM dsRNA-FITC (85 μl SFM+5μ 20 mM dsRNA-FITC+10 μl 400 mM MSM) mixed well and incubated at 37° C. The results are shown in FIG. 3. FIG. 3 shows the delivery of a dsRNA-FITC into the neuroblastoma cell where the dsRNA is delivered in the presence of MSM. The MSM was premixed with the RNA-FITC for 30 minutes. As can be seen from the increased number of distinct spots, the dsRNA-FITC transcript has passed through a number of cell membranes and entered a greater number of cells;

FIG. 3B is a photograph of three panels A, B, and C of the neuroblastoma cell as prepared in accordance with b. above. The panels show the delivery of a dsRNA-FITC into the neuroblastoma cell where the dsRNA is delivered in the absence of MSM. Incubation time was overnight.

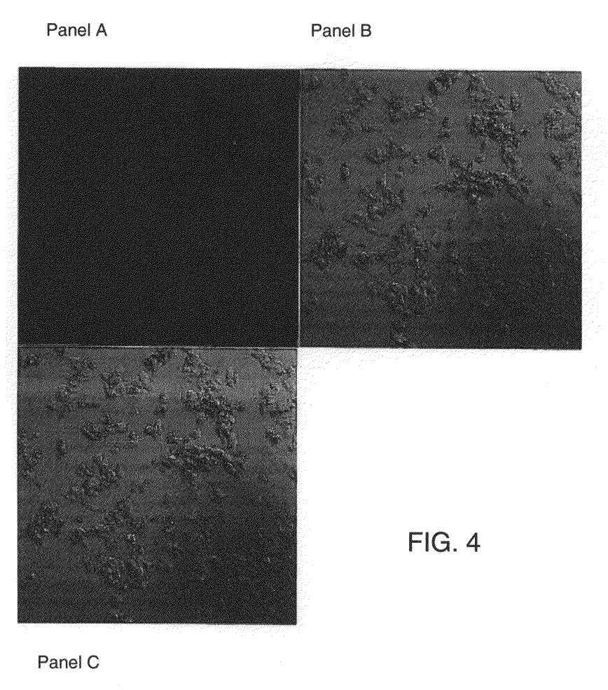

FIG. 4 is a photograph of three, panels A, B, and C of the neuroblastoma cells as prepared in accordance with c. above. The panels show the delivery of a dsRNA-FITC into the neuroblastoma cell where the dsRNA is delivered in the presence of MSM. The MSM was premixed with the RNA-FTC for 30 minutes. The presence of the dsRNA-FITC in the neuroblastoma cell is clearly indicated.

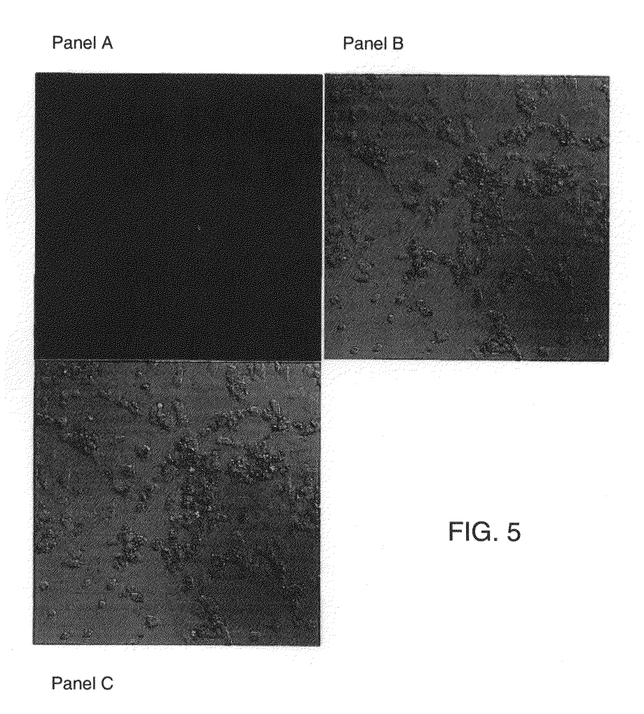

FIG. 5 is a photograph of three panels A, B, and C of the neuroblastoma cell prepared in accordance with d. above. The panels show the delivery of a dsRNAFITC into the neuroblastoma cell where the dsRNA is delivered in the presence of MSM. The MSM was premixed with the RNA-FTC for 30 minutes. An even greater presence of the dsRNA-FITC in the neuroblastoma cell is clearly indicated.

### Example II

### siRNA Delivery

In accordance with the methods of the disclosure, human neuroblastma cells (SH-SY5Y) were prepared and cultured as indicated above. Specifically, the SH-SY5Y cells (0.25×106) with 1 ml complete medium were seeded in each well of two 4-well chamber slides, incubated at 37° C. for 4 hours to let the cells adhere to slides and to become about 80% confluent. Photographs of the neuroblastoma cells were taken with a fluorescent microscope and the results are shown below.

Assignment:

a. Control: Untreated cells (100 μl SFM);

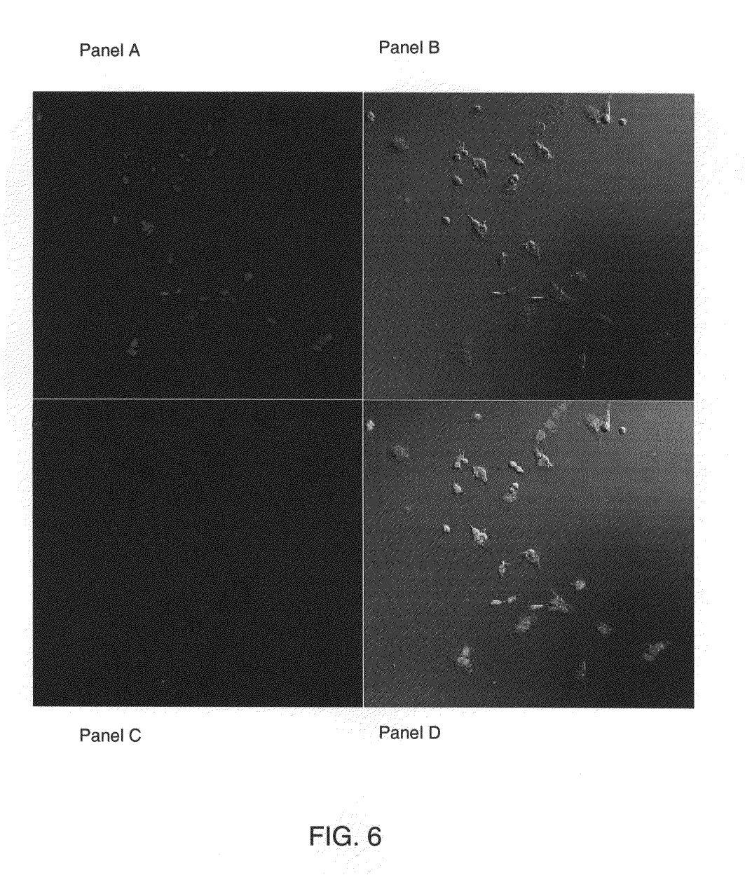

Without MSM, added 100 nM Cy3-SiRNA: (95 μl SFM, Serum free medium+5 μl 20 mM Cy3). These results are shown in FIG. 6. FIG. 6 shows four panels A, B, C, and D of neuroblastoma cells wherein the cells have been contacted with a CY3-SiRNA (Red Nuclear stain, Syto16:green stain) construct in the absence of MSM;

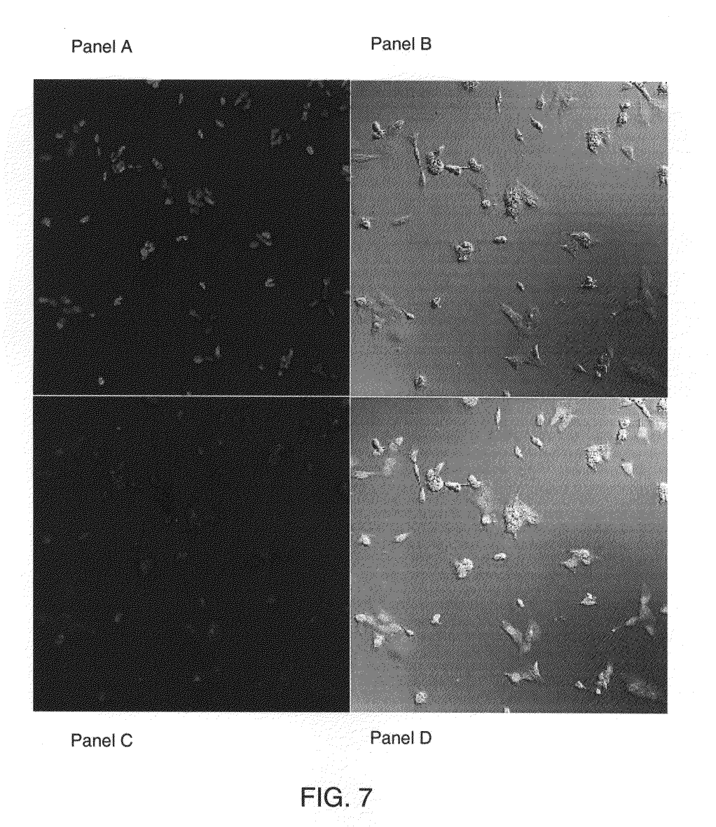

c. With 4 mM MSM, added 100 nM Cy3-SiRNA (85 μl SFM+5 μl 20 mM Cy3+10 μl 400 mM MSM) mixed well and incubated at RT for 30 minutes. These results are shown in FIG. 7. FIG. 7 shows four panels A, B, C, and D of neuroblastoma cells wherein the cells have been contacted with a CY3-dsRNA (Red Nuclear stain, Syto16:green stain) construct in the presence of MSM. The presence of the Cy3-SiRNA in the neuroblastoma cell is clearly indicated;

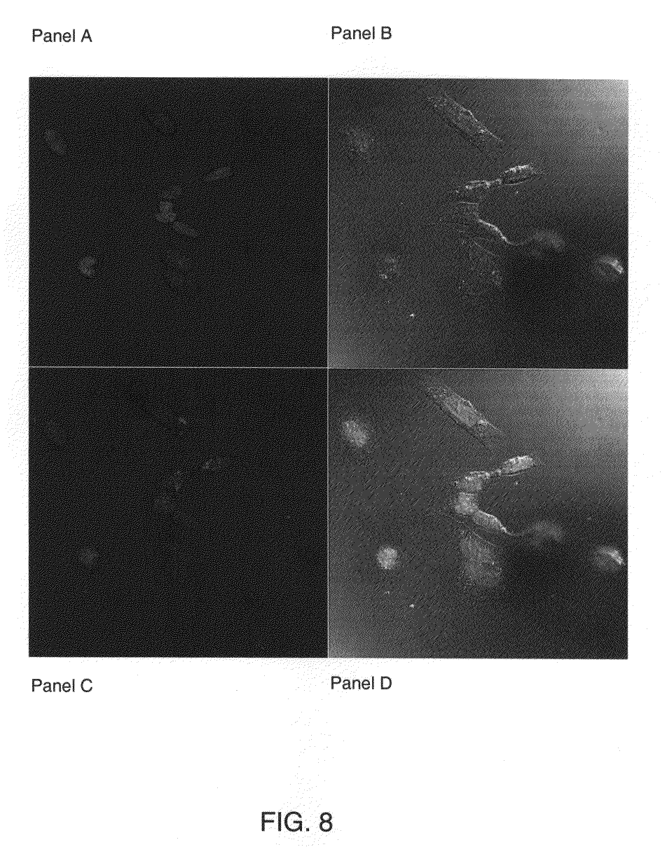

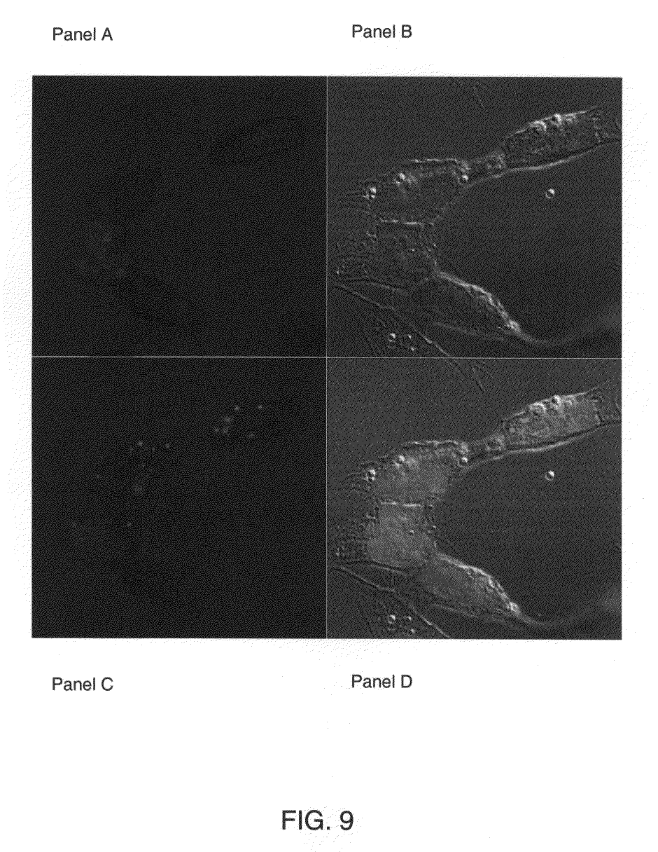

d. With 4 mM MSM, added 100 nM Cy3-SiRNA (85 μl SFM+5 μl 20 mM Cy3+10 μl 400 mM MSM) mixed well and incubated at 37° C. for 60 minutes. These results are shown in FIG. 8. FIG. 8 shows four panels A, B, C, and D of neuroblastoma cells wherein the cells have been contacted with a CY3-dsRNA (Red Nuclear stain, Syto16:green stain) construct in the presence of MSM. The increased and distinct presence of the Cy3-SiRNA in the neuroblastoma cell is clearly indicated. Zoom indicated location of Cy3 dsRNA. Additionally, FIG. 9 is a zoomed in photograph of the four panels A, B, C, and D of the neuroblastoma cell where the dsRNA is delivered in the presence of MSM. The zoom indicates the location of Cy3 dsRNA in single cells.

### Example III

### Antibody Delivery

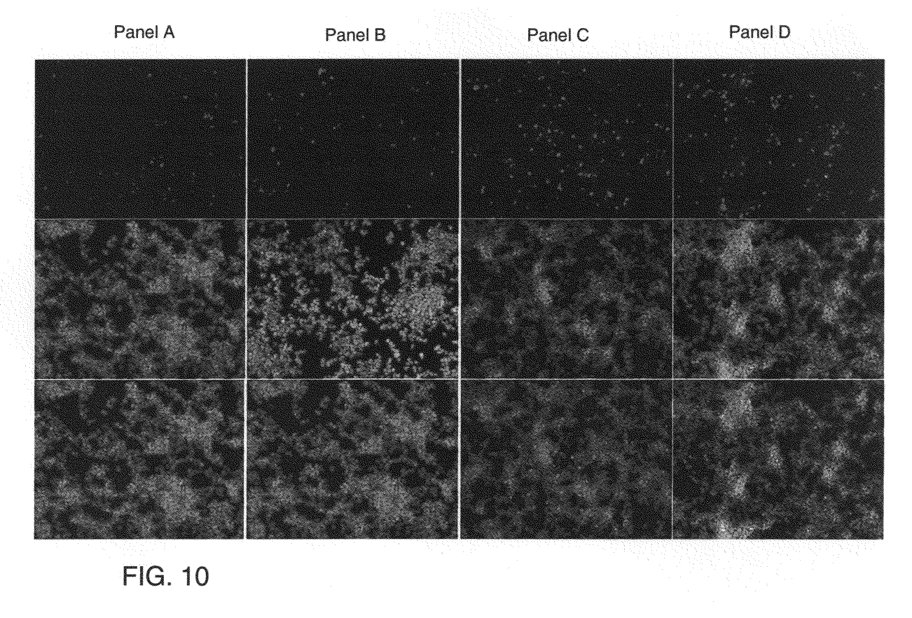

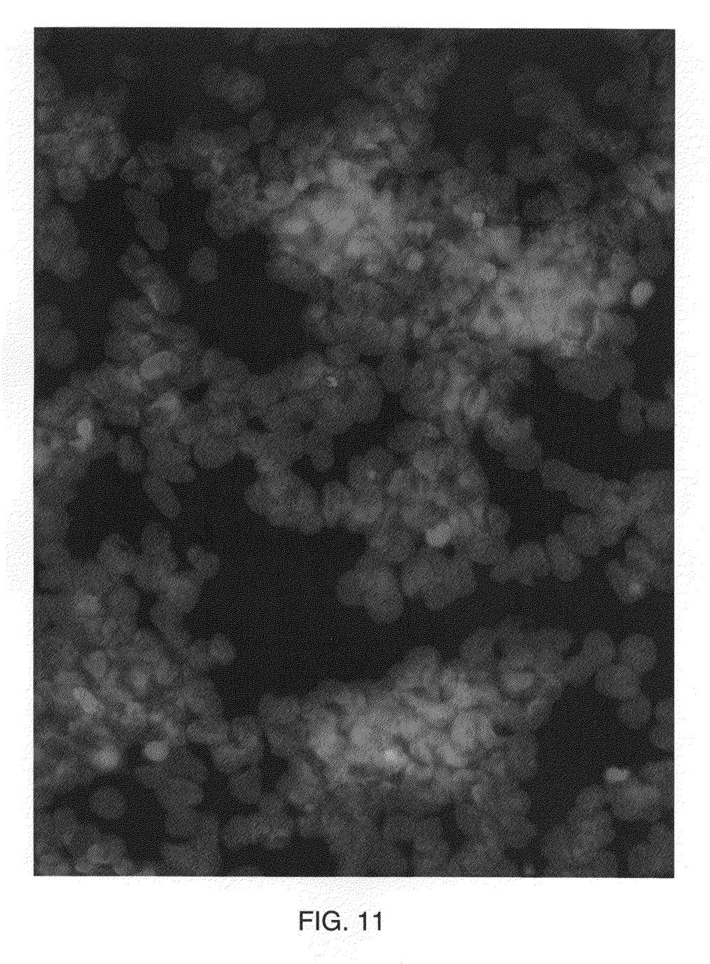

In accordance with the methods of the disclosure, human neuroblastma cells (SH-SY5Y) were prepared and cultured as indicated above. Specifically, the SH-SY5Y cells (0.25×106) with 1 ml complete medium were seeded in each well of two 4-well chamber slides, incubated at 37° C. for 4 hours to let the cells adhere to slides and to become about 80% confluent. Photographs of the neuroblastoma cells were taken with a fluorescent microscope and the results are shown below with respect to FIGS. 10 and 11.

For the antibody delivery experiments, the method was essentially the same with the following differences. Instead of dsRNA, Rhodamine-conjugated IgG (rabbit) was used. Premixing time was 30 min at 37° C. Delivery time was 1-4 hrs.

FIG. 10 is a photograph of 4 panels (A, B, C, and D) of six slides of SHSY5Y cells taken with a fluorescent microscope showing the delivery of a secondary antibody conjugated to Texas red into the SH-SY5Y cells (counterstained with DAPI) where the AB is delivered either in the absence or in the presence of MSM. Incubation time was 1 hour. Panel A was AB alone, panel B was AB+MSM (0.4 mM), panel C was AB+MSM (4 mM), and panel D was AB+MSM (4 mM), wherein before adding the AB and/or MSM into the cell culture medium the AB and MSM were mixed well and incubated at 37° for 30 mins. Magnification was at 200×. As can be seen, the increased fluorescence show that the AB+MSM is effectively delivered to the target cells of interest.

FIG. 11 is a photograph of a single panel of SH-SY5Y cells taken with a fluorescent microscope showing the delivery of a secondary antibody conjugated to Texas red into the SH-SY5Y cells (counterstained with DAPI) where the dsRNA is delivered in the presence of MSM. Incubation time was 1 hour. Before adding the AB and/or MSM into the cell culture medium the AB and MSM were mixed well and incubated at 37° for 30 mins. Magnification was at 400×. Again, the increased fluorescence indicates that the AB+MSM is effectively delivered to the target cells of interest.

### Example IV

### dsRNA Delivery II

In accordance with the methods of the disclosure, human neuroblastma cells (SH-SY5Y) were prepared and cultured as indicated above. Specifically, the SH-SY5Y cells (0.25×106) with 1 ml complete medium were seeded in each well of two 4-well chamber slides, incubated at 37° C. for 4 hours to let the cells adhere to slides and to become about 80% confluent. Photographs of the neuroblastoma cells were taken with a fluorescent microscope and the results are shown below.

Assignment:

Panel A. Control: Untreated cells (100 μl SFM);

Panel B. Without MSM, added 100 nM Cy3-SiRNA: (95 μl SFM, Serum free medium+5 μl 20 mM Cy3);

Panel C. With 4 mM MSM, added 100 nM Cy3-SiRNA (85 μl SFM+5 μl 20 mM Cy3+10 μl 400 mM MSM) mixed well and incubated at RT for 30 minutes, and

Panel D. With 4 mM MSM, added 100 nM Cy3-SiRNA (85 μl SFM+5 μl 20 mM Cy3+10 μl 400 mM MSM) mixed well and incubated at 37° C. for 60 minutes.

Not Shown: Panel E. With FuGENE 6 Transfection Reagent added 100 nM Cy3-SiRNA and (80 μl SFM+5 μl 20 mM Cy3+15 μl Transfection reagent) mixed well and incubated at RT for 30 minutes.

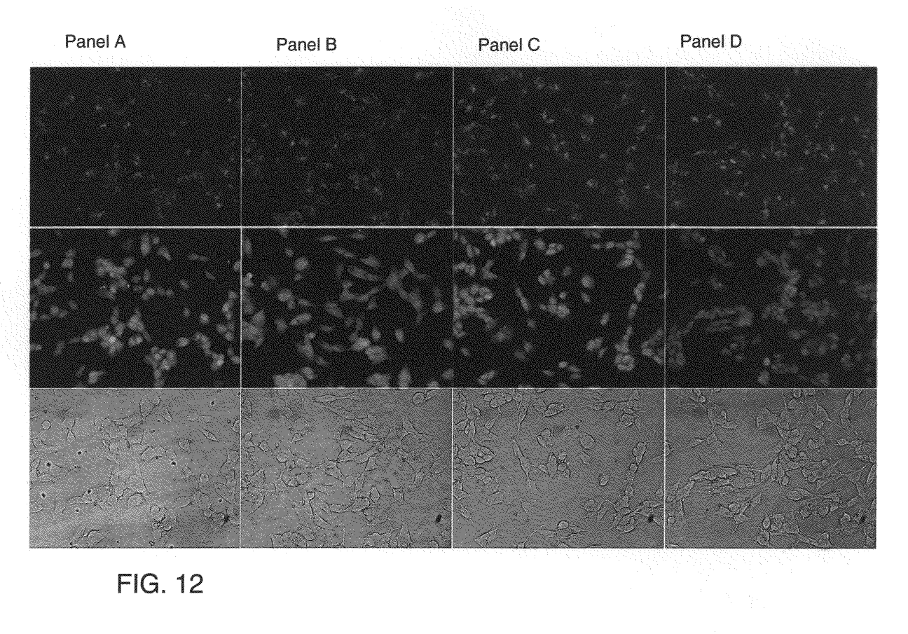

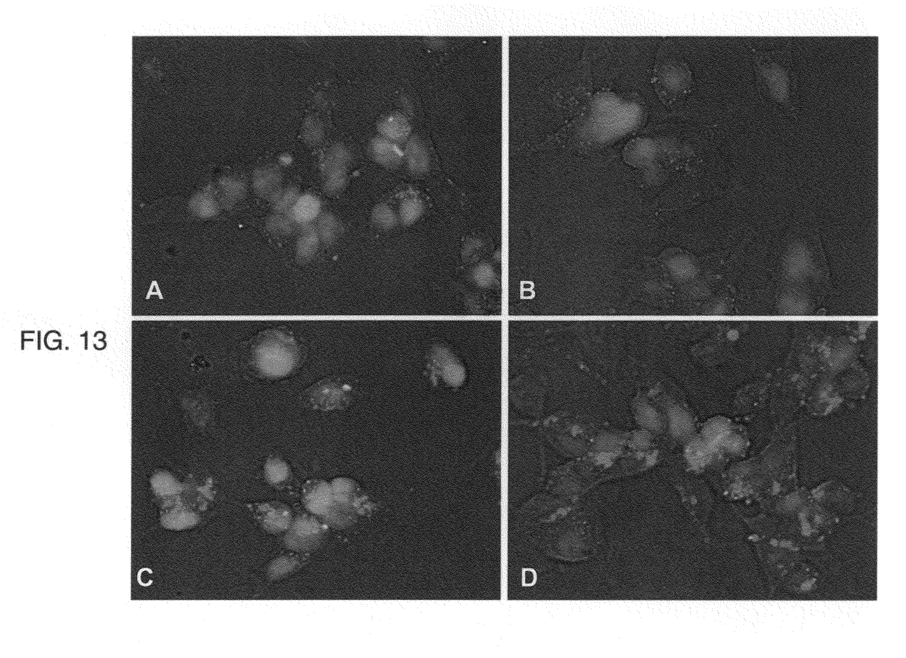

The solutions (a-e) were added into proper wells. Then 900 μl of complete medium was added to all the chambers and the slides were placed in the 37° C. incubator for overnight. As indicated above, Syto16 Green was prepared (1:100, 10 μM)−792 μl of SFM+8 μl of Syto16 (1.0 mM), mixed well, and added to each well and incubated at 37° C. for 30 minutes. The medium with the dyes was removed from chamber slides. Washed with PBS three times. Fixed all slides in 10% Paraformaldehyde at RT for 10 minutes, washed with PBS again. The slides were preserved with mounting medium. The slides were observed under fluorescent light microscope and the results are shown in FIGS. 12 and 13.

FIG. 12 is a photograph of four wells, panels A, B, C, and D of a neuroblastoma cell taken with a fluorescent microscope showing the delivery of a CY3-dsRNA (Red Nuclear stain, Syto16:green stain) into the neuroblastoma cell where the dsRNA is delivered either in the presence or absence of MSM. Incubation time was over night. Magnification was at 400×. In agreement with the results indicated above, a greater presence of the CY3-dsRNA is shown in the neuroblastoma cells wherein the composition included MSM in addition to CY3-dsRNA.

FIG. 13 is a photograph of four wells, panels A, B, C, and D of a neuroblastoma cell taken with a fluorescent microscope showing the delivery of a CY3-dsRNA (Red Nuclear stain, Syto16:green stain) into the neuroblastoma cell where the dsRNA is delivered either in the presence or absence of MSM. Incubation time was over night. Magnification was at 400×. Again the results indicate a greater presence of the CY3-dsRNA is shown in the neuroblastoma cells wherein the composition included MSM in addition to CY3-dsRNA.

### Example V

### Cyclosporin Delivery in Rats

Materials and methods: MSM, purchased from Sigma. ³H cyclosporin, purchased from American Radiolabeled Chemicals, Inc. Biosol biodegradable tissue solubilizer, purchased from National Diagnostics, Inc. (Atlanta, Ga.). UniScint BD, a biodegradable liquid scintillator for counting high salt samples when used in combination with Biosol, also purchased from National Diagnostics, Inc.

Solutions prepared: (A) MSM, PBS, ³H cyclosporin, 2:1:1 (40 μl 10% MSM, 20 μl PBS, 20 μl ³H cyclosporin); (B) PBS, ³H cyclosporin, 3:1 (60 μl PBS, 20 μl ³H cyclosporin).

All procedures were carried out one rat at a time: weighing; administering anesthesia via IP injection of ketamine (80 mg/kg) and xylazine (5 mg/kg); injecting with 10 μl of solution A or B, at 10, 15, and 30 minutes; sacrificing the rat; removing eyeball and washing 2× with 2 ml PBS; removal of the aqueous from the eye, about 10 μl, with a 31 gauge insulin syringe, followed by dispersion in a 100 μl tube with PBS; dissecting eyeball and placing the vitreous, retina, and choroid into a 100 μl tube containing PBS; transferring into scintillation counting vial with 1 ml of Biosol to digest separately; placing samples into a 50 degrees C. water bath for three hours with 20 rpm gentle shaking; adding 10 ml of counting fluid to each counting vial, followed by gentle mixing; counting ³H cyclosporin using Protocol No. 10 of Perkin-Elmer scintillation counter.

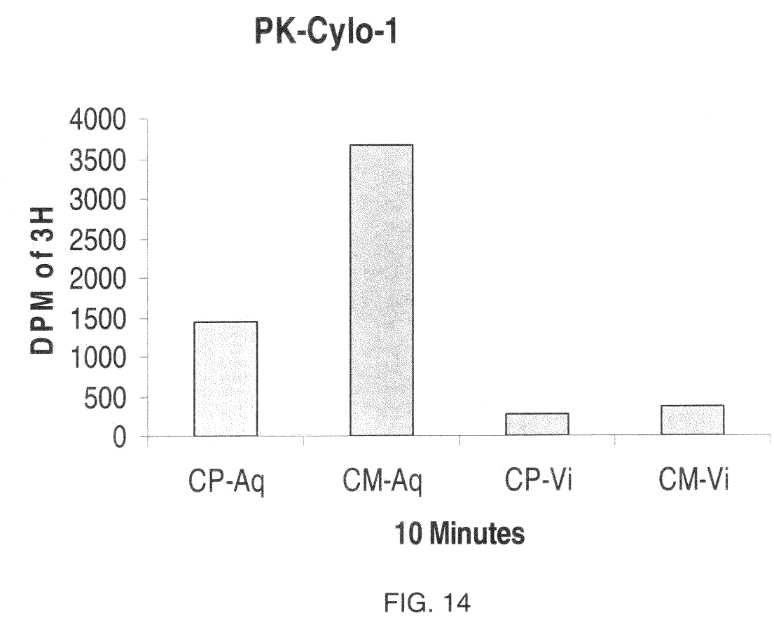

The results are illustrated in FIGS. 14-18:

FIG. 14 shows the relative quantities of ³H cyclosporin in the aqueous obtained with solution A (containing MSM; “CM-Aq” in the figure) and with solution B (no MSM; “CP-Aq” in the figure). As may be seen, the presence of MSM significantly increased the concentration of cyclosporin in the aqueous. FIG. 14 also shows the relative quantities of ³H cyclosporin in the vitreous obtained with solution A (containing MSM; “CM-Vi” in the figure) and with solution B (no MSM; “CP-Vi” in the figure). An increase in the amount of cyclosporin in the vitreous was also seen, although the increase was somewhat less marked than for the aqueous.

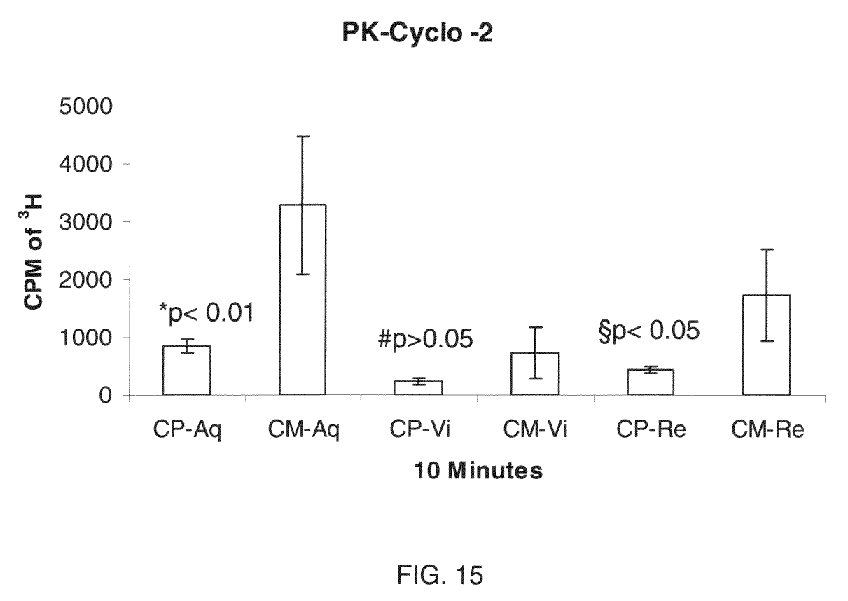

FIG. 15 also illustrates the relative quantities of ³H cyclosporin delivered into the aqueous and the vitreous with and without MSM, and further illustrates the relative quantities delivered into the retina with MSM (“CM-Re”) and without MSM (“CP-Re”). Again, a marked increase is seen with the MSM-containing cyclosporin formulation. The P values are CP-Aqueous vs CM-Aqueous=0.00634; CP-Vitreous vs CM-Vitreous p=0.069133; CP-Retina/Choroid vs CM-Retina/Choroid p=0.017007.

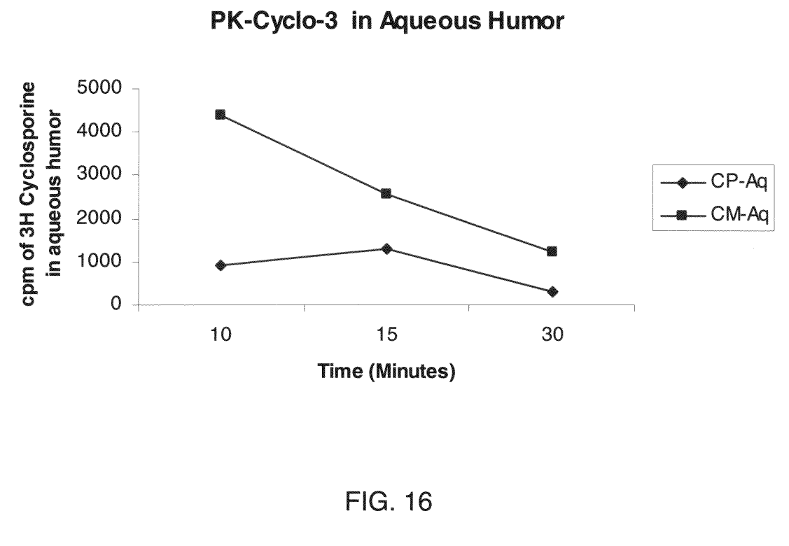

FIG. 16 is a graph illustrating the amount of cyclosporin delivered to the aqueous in the presence and absence of MSM, with the 10 minute, 15 minute, and 30 minute data points shown.

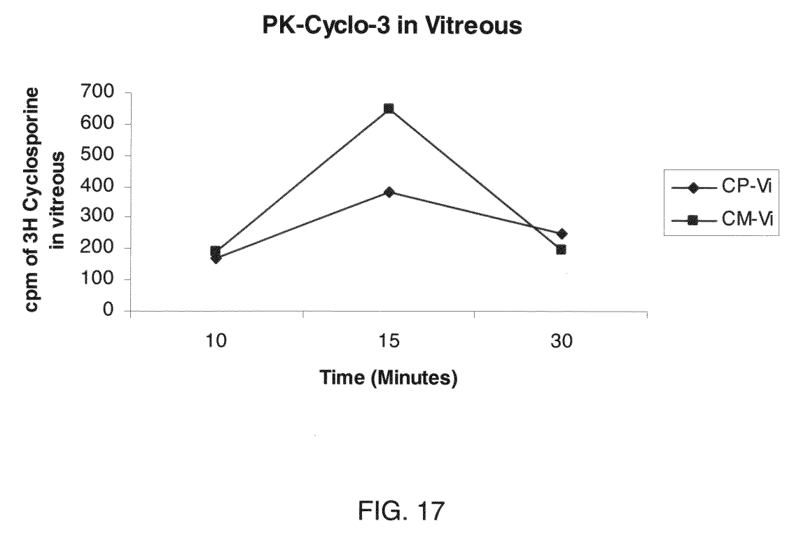

FIG. 17, similarly, is a graph illustrating the amount of cyclosporin delivered to the vitreous in the presence and absence of MSM, with the 10 minute, 15 minute, and 30 minute data points shown.

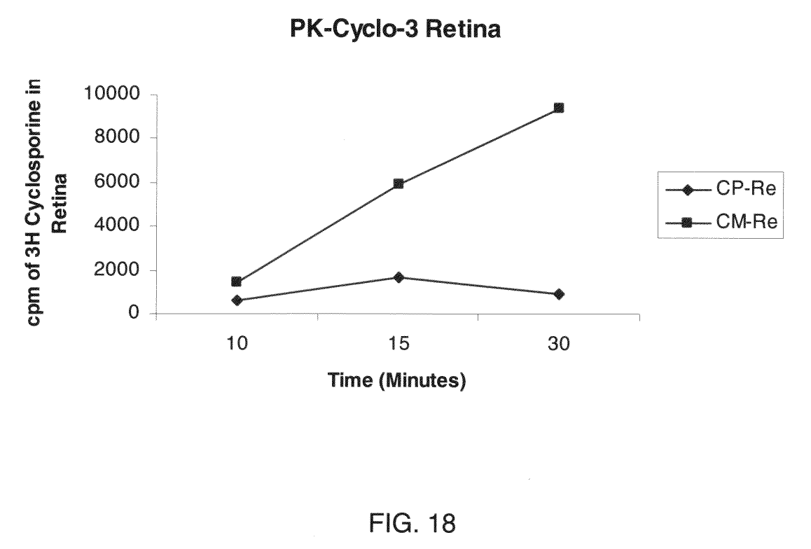

FIG. 18, similarly, is a graph illustrating the amount of cyclosporin reaching the retina in the presence and absence of MSM, with the 10 minute, 15 minute, and 30 minute data points shown. When the cyclosporin is administered in an MSM-containing formulation, it should be noted that the amount of cyclosporin reaching the retina increases with time.

### Example VI

### Bevacizumab Delivery

In this experiment, the ability of MSM to facilitate delivery of bevacizumab, a monoclonal antibody (Avastin®, Genentech/Roche), to the vitreous, retina, and choroid when co-applied to the rat eye.

Control: bevacizumab, 12.5 mg/ml in saline. Experimental: bevacizumab, 12.5 mg/ml, in 5% MSM.

Ten μl of solution was administered to each eye of the rats, one rat at a time (procedures analogous to those used in the preceding example). Bevacizumab was quantified in various components of the rat eyes using an ELISA technique.

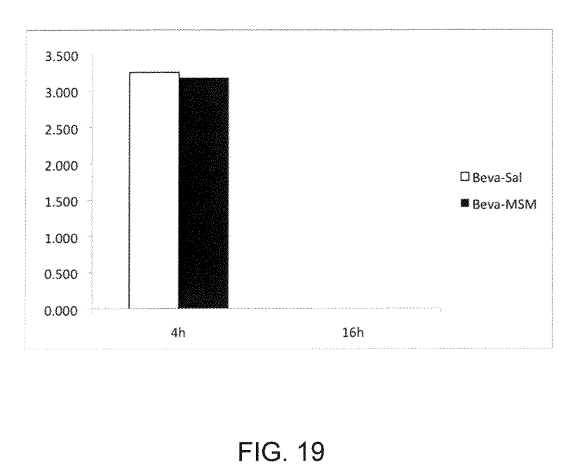

FIG. 19 illustrates the relative levels of bevacizumab in the retina/choroid with and without MSM (i.e., using the experimental and control solutions) using a single dose and evaluating bevacizumab levels after 4 hours and 16 hours; numbers given are μg bevacizumab per gm protein.

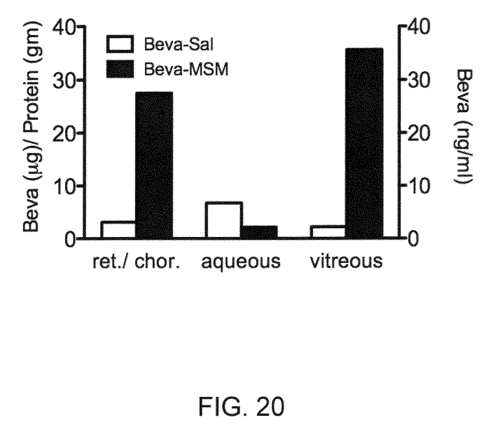

FIG. 20 illustrates bevacizumab levels in the retina/choroid vitreous humor (again, numbers given are μg bevacizumab per gm protein) following multiple doses of the control and experimental solutions, with bevacizumab levels evaluated after 4 hours.

| | | Aqueous | Vitreous |
|---|---|---|---|
| | Retina-choroid | Humor | Humor |
| | (μg/gm prot) | (ng/ml) | (ng/ml) |
| Beva-Sal | 3.11 | 6.75 | 2.22 |
| Beva-MSM | 27.50 | 2.14 | 35.64 |

All publications and patents cited in this specification are hereby incorporated by reference herein.

While the invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the invention. All such modifications are intended to be within the scope of the claims appended hereto.

We claim:

1. A method for treating a subject afflicted with an adverse condition, comprising administering to the subject a formulation comprising an effective transport-enhancing amount of methylsulfonylmethane and a therapeutically effective amount of a nucleic acid.

2. The method of claim 1, wherein the nucleic acid is a ribonucleic acid.

3. The method of claim 2, wherein the ribonucleic acid is selected from messenger RNA, transfer RNA, ribosomal RNA, interfering RNA, small nuclear RNA, and anti-sense RNA.

4. The method of claim 2, wherein the ribonucleic acid is single-stranded or double-stranded.

5. The method of claim 4, wherein the ribonucleic acid is double-stranded.

6. The method of claim 1, wherein the nucleic acid is a deoxyribonucleic acid.

7. The method of claim 1, wherein the deoxyribonucleic acid is selected from linear DNA, cDNA, plasmid DNA, chromosomal DNA, and viral DNA.

8. The method of claim 6, wherein the deoxyribonucleic acid is single-stranded or double-stranded.

9. The method of claim 1, wherein the nucleic acid comprises a single nucleotide polymorphism, an expressed sequence tag, or both a single nucleotide polymorphism and an expressed sequence tag.

10. The method of claim 1, wherein the nucleic acid is an aptamer.

11. The method of claim 1, wherein the methylsulfonylmethane is present in an amount in the range of about 0.01 wt. % to about 10 wt. %.

12. The method of claim 1, wherein the methylsulfonylmethane is present in an amount in the range of about 1 wt. % to about 6 wt. %.

13. The method of claim 1, wherein administration is enteral or parenteral.

14. The method of claim 1, wherein administration is ophthalmic.

15. The method of claim 1, wherein the nucleic acid comprises a transcription cassette.

16. The method of claim 1, wherein the adverse condition is the result of an overexpression of a protein.

17. The method of claim 1, wherein the adverse condition is the result of a protein deficiency.

18. A method for treating a subject afflicted with an adverse condition, comprising administering to the subject a formulation comprising an effective transport-enhancing amount of methylsulfonylmethane and a therapeutically effective amount of a peptidic agent.

19. The method of claim 18, wherein the peptidic agent is selected from oligopeptides, polypeptides, proteins, and protein fragments.

20. The method of claim 18, wherein the peptidic agent is selected from a transcription factor, an enzyme, an antibody, an antibody fragment, an antigen, a coagulation modulator, a cytokine, an endorphin, a peptidic hormone, and combinations thereof.

21. The method of claim 18, wherein the peptidic agent is an antibody or fragment thereof.

22. The method of claim 21, wherein the antibody is selected from Abciximab, Adalimumab, Alemtuzumab, Basiliximab, Bevacizumab, Cetuximab, Dacilzumab, Eculizumab, Efalizumab, Etanerecept, Gemtuzumab, Ozogamicin, Ibritumomab tiuxetan, Infliximab, Muromonab-CD3, Natalizumab, Omalizumab, Palivizumab, Panitumumab, Ranibizumab, Rituximab, Tositumomab, Trastuzumab, and combinations thereof.

23. The method of claim 22, wherein the antibody is Bevacizumab.

24. The method of claim 18, wherein the methylsulfonylmethane is present in an amount in the range of about 0.01 wt. % to about 10 wt. %.

25. The method of claim 24, wherein the methylsulfonylmethane is present in an amount in the range of about 1 wt. % to about 6 wt. %.

26. The method of claim 18, wherein administration is enteral or parenteral.

27. The method of claim 18, wherein administration is ophthalmic.

28. A method for treating a subject afflicted with an adverse condition, comprising systemically administering to the subject a formulation comprising:
an effective transport-enhancing amount of a compound having the structure of formula (I) 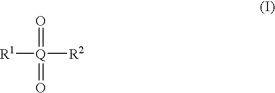
wherein R¹ and R² are independently selected from C₁-C₆ alkyl, C₁-C₆ heteroalkyl, C₆-C₁₄ aralkyl, and C₂-C₁₂ heteroaralkyl, any of which may or may not be substituted, and Q is S or P;
a therapeutically effective amount of a biologically active agent that has a molecular weight of at least 300; and
a pharmaceutically acceptable carrier.

29. The method of claim 28, wherein the biologically active agent is a macromolecular biomolecule selected from nucleic acids, peptidic compounds, polysaccharides, lipopolysaccharides, and lipids.

30. The method of claim 28, wherein the methylsulfonylmethane is present in an amount in the range of about 1 wt. % to about 8 wt. %.

31. The method of claim 30, wherein the methylsulfonylmethane is present in an amount in the range of about 2 wt. % to about 5 wt. %.

32. The method of claim 26, wherein administration is enteral or parenteral.

33. A pharmaceutical formulation comprising about 1 wt. % to about 8 wt. % methylsulfonylmethane and a therapeutically effective amount of a biologically active agent selected from nucleic acids, peptidic agents, polysaccharides, lipopolysaccharides, lipids, and combinations thereof.
